(19) United States
(12) Patent Application Publication
(10) Pub. No.: US 2012/0130137 A1
(43) Pub. Date: May 24, 2012

(54) PRODUCTION PROCESS OF CONJUGATED DIENE

(75) Inventors: Souichi ORITA, Okayama (JP); Hiroshi Takeo, Okayama (JP); Masaru Utsunomiya, Mie (JP); Takuma Nishio, Mie (JP); Hiroyuki Yagi, Mie (JP); Nariyasu Kanuka, Mie (JP)

(73) Assignee: MITSUBISHI CHEMICAL CORPORATION, Tokyo (JP)

(21) Appl. No.: 13/305,078

(22) Filed: Nov. 28, 2011

Related U.S. Application Data
(63) Continuation of application No. PCT/JP2010/058842, filed on May 25, 2010.

(30) Foreign Application Priority Data
May 29, 2009 (JP) 2009-131147

(51) Int. Cl. C07C 5/48

(52) U.S. Cl. 585/621

(57) ABSTRACT

The present invention relates to a process of producing a conjugated diene including a step of mixing a raw material gas containing a monoolefin having a carbon atom number of 4 or more with a molecular oxygen-containing gas and supplying the mixture into a reactor, and a step of obtaining a corresponding conjugated diene-containing product gas produced by the oxidative dehydrogenation reaction of the monoolefin having a carbon atom number of 4 or more in the presence of a catalyst, wherein the concentration of a combustible gas in the gas supplied to the reactor is not less than the upper explosion limit and the oxygen concentration in the product gas is from 2.5 to 8.0 vol %.

### TECHNICAL FIELD

The present invention relates to a production process of a conjugated diene. More specifically, the present invention relates to a process for producing a conjugated diene such as butadiene by a catalytic oxidative dehydrogenation reaction of a monoolefin having a carbon atom number of 4 to more, such as n-butene.

### BACKGROUND ART

The process for producing a conjugated diene such as butadiene (hereinafter, sometimes referred to as “BD”) by an oxidative dehydrogenation reaction of a monoolefin such as n-butene in the presence of a catalyst includes, for example, a catalytic oxidative dehydrogenation reaction according to the following reaction formula. In this reaction, water is by-produced.

C₄H₈+½O₂→C₄H₆+H₂O

As an industrial process for producing butadiene by the catalytic oxidative dehydrogeneration reaction, there has been proposed a method where a mixture containing 1-butene as well as 2-butene and the like, obtained by separating butadiene in an extractive distillation column in the process of extracting and separating butadiene from a C₄ fraction (a mixture of hydrocarbons having a carbon atom number of 4; hereinafter, sometimes referred to as “BB”) produced as a byproduct in naphtha cracking (hereinafter, this mixture is sometimes referred to as “BBSS”) is used as a raw material and butadiene is produced from butenes contained in the BBSS.

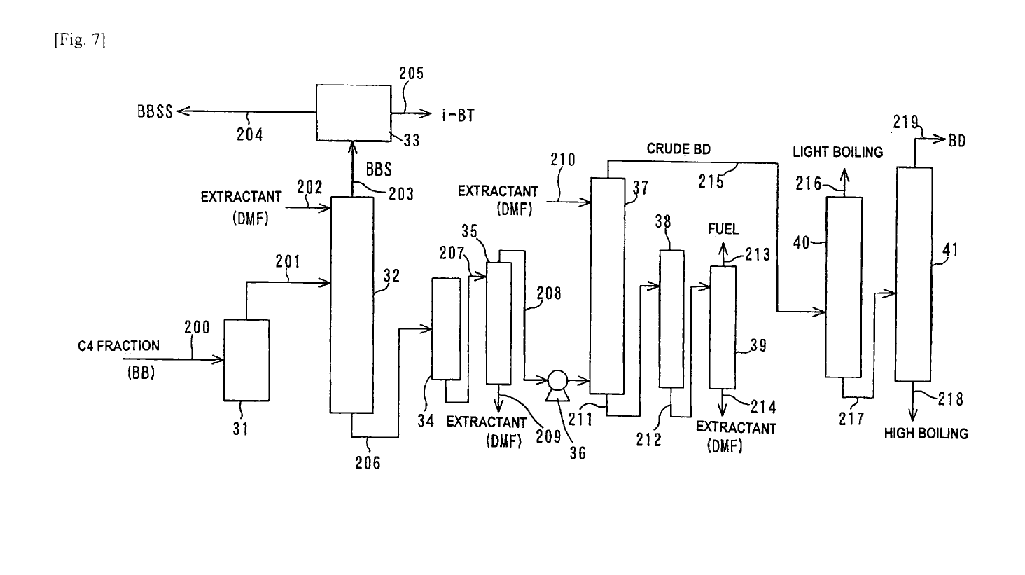

The representative process as the extraction and separation process of butadiene from a C₄ fraction includes, for example, the process shown in FIG. 7. First, a C₄ fraction is introduced into a first extractive distillation column 32 through an evaporation column 31, where butadiene and the like are extracted with an extractant (e.g., dimethylformamide (DMF)) and at the same time, other C₄ components (hereinafter, sometimes referred to as “BBS”) is removed by evaporation. As for BBS, i-butene is subsequently removed in an i-butene separation column 33, and BBSS is discharged out of the system.

The butadiene extract from the first extractive distillation column 32 flows to a preliminary stripping column 34 and a first stripping column 35, where the extractant such as DMF is removed, and then is introduced through a compressor 36 into a second extractive distillation column 37 and again subjected to extraction with an extractant (e.g., DMF). Acetylenes separated in the second extractive distillation column 37 are recovered as a fuel through a butadiene recovery column 38 and a second stripping column 39.

The crude BD from the second extractive distillation column 37 is further purified in a first distillation column 40 and a second distillation column 41, and high-purity 1,3-butadiene is recovered. In FIG. 7, numerals 200 to 219 indicate piping.

As a representative process for producing butadiene by the above-described catalytic oxidative dehydrogenation reaction of n-butene, Patent Document 1 has proposed the following production process of butadiene:

(1) a reaction step of producing butadiene by a gas-phase catalytic oxidative dehydrogenation of n-butene,

(2) a cooling step of cooling the product gas obtained from the reaction step to remove trace high-boiling-point byproducts contained in the product gas,

(3) an aldehyde removing step of removing a small amount of aldehydes contained in the cooled product gas,

(4) a compression step of compressing the guided product gas, and

(5) a C₄ recovery step of recovering C₄ components containing butadiene and other C₄ hydrocarbons from the compressed product gas.

The composite oxide catalyst used in the catalytic oxidative dehydrogenation reaction of n-butene includes, for example, the catalyst described in Patent Document 2, where a composite oxide catalyst containing silica and at least one member of molybdenum, iron, nickel and cobalt is described but the production process of butadiene is not specifically described.

### RELATED ART

### Patent Document

Patent Document 1: JP-A-60-115532

Patent Document 2: JP-A-2003-220335

### SUMMARY OF THE INVENTION

### Problems That the Invention is to Solve

Patent Documents 1 and 2 are silent on the method to avoid an explosion when recovering hydrocarbons containing butadiene from the product gas by using a solvent after producing butadiene by an oxidative dehydrogenation reaction of butene, but the oxidative dehydrogenation reaction uses a combustible gas such as raw material hydrocarbon and an oxygen-containing gas and therefore, an explosion during reaction must be avoided. As one method to avoid an explosion, it may be considered to deviate the combustible gas concentration in the gas from the explosion range determined by the combustible gas composition, oxygen and an inert gas. In this case, there may be further considered two cases, that is, a case where the combustible gas concentration is set to be not more than a lower explosion limit, and a case where the concentration is set to be not less than an upper explosion limit. In the case of not more than a lower explosion limit, the raw material gas concentration is low and for practicing the reaction in industry, this is disadvantageous in view of efficiency and profitability. Therefore, a reaction at a concentration not less than an upper explosion limit is preferred.

In the case where the reaction is performed using a gas having a combustible gas concentration not less than the upper explosion limit, the reaction step is outside the explosion range and the reaction safely proceeds, but when the product gas is contacted with an absorption solvent to let the product hydrocarbon be absorbed by the solvent, the combustible gas concentration that has been not less than the upper explosion limit decreases, as a result, the product gas composition traverses the explosion range, leading to a high probability of explosion in a later step after the reaction step. Furthermore, at the time of producing butadiene by an oxidative dehydrogenation reaction of butene in the presence of a catalyst, if the oxygen concentration in the gas is too low, coking of a carbon portion or the like proceeds on the catalyst to increase the differential pressure in the reactor and this may cause a trouble in continuing the operation. On the other hand, if the oxygen concentration in the gas is too high, this is found to incur a problem that many high-boiling-point byproducts are produced and allowed to be contained in the product gas and when the product gas containing these high-boiling-point byproducts is cooled in the later cooling step, a solid matter attributable to the high-boiling-point byproduct in the product gas is precipitated during the cooling step, as a result, clogging occurs in the cooling step to cause a trouble in continuing the operation.

The present invention has been made by taking these problems into consideration, and an object of the present invention is to provide a process for producing a conjugated diene such as butadiene by a catalytic oxidative dehydrogenation reaction of a monoolefin such as n-butene, ensuring that coking on a catalyst is suppressed when continuously using a catalyst, the amount of high-boiling-point byproducts produced is reduced, and production of a conjugated diene such as butadiene can be more safely and stably performed with a high yield.

### Means for Solving the Problems

That is, the present invention relates to the following production process of a conjugated diene.

<1> A production process of a conjugated diene, comprising a step of mixing a raw material gas containing a monoolefin having a carbon atom number of 4 or more and a molecular oxygen-containing gas and supplying the mixture to a reactor, and a step of obtaining a corresponding conjugated diene-containing product gas produced by an oxidative dehydrogenation reaction of the monoolefin having a carbon atom number of 4 or more in the presence of a catalyst, wherein the concentration of a combustible gas in the gas supplied to the reactor is not less than the upper explosion limit and the oxygen concentration in the product gas is from 2.5 to 8.0 vol %.

<2> The production process of a conjugated diene as described in <1> above, which further comprises a step of bringing the conjugated diene-containing product gas into contact with an absorption solvent to obtain a conjugated diene-containing solvent.

<3> The production process of a conjugated diene as described in <1> or <2> above, wherein the catalyst is a composite oxide catalyst containing at least molybdenum, bismuth and cobalt.

<4> The production process of a conjugated diene as described in <3> above, wherein the catalyst is a composite oxide catalyst represented by the following formula (1):

MoₐBi_{b}Co_{c}Ni_{d}FeₑX_{f}Y_{g}ZₕSiᵢOⱼ (1)

(wherein X is at least one element selected from the group consisting of magnesium (Mg), calcium (Ca), zinc (Zn), cerium (Ce) and samarium (Sm), Y is at least one element selected from the group consisting of sodium (Na), potassium (K), rubidium (Rb), cesium (Cs) and thallium (Tl), Z is at least one element selected from the group consisting of boron (B), phosphorus (P), arsenic (As) and tungsten (W), a to j represent an atomic ratio of respective elements and when a=12, are in ranges of b=0.5 to 7, c=0 to 10, d=0 to 10 (provided that c+d=1 to 10), e=0.05 to 3, f=0 to 2, g=0.04 to 2, h=0 to 3 and i=5 to 48, and j is a numerical value satisfying the oxidation state of other elements).

<5> The production process of a conjugated diene as described in <4> above, wherein the composite oxide catalyst is a catalyst produced through a step including integration in an aqueous system and heating of supply source compounds of respective component elements constituting the composite oxide catalyst and is produced by a method comprising a pre-step of producing a catalyst precursor by heat-treating an aqueous solution or aqueous water dispersion of the raw material compound containing silica and at least one member selected from the group consisting of a molybdenum compound, an iron compound, a nickel compound and a cobalt compound, or a dry matter resulting from drying of the aqueous solution or aqueous water dispersion, and a post-step of integrating the catalyst precursor, a molybdenum compound and a bismuth compound together with an aqueous solvent, and drying and firing the mixture.

<6> The production process of a conjugated diene as described in any one of <1> to <5> above, wherein the oxygen concentration of the product gas is measured at the outlet of the reactor and at least either one of the amount of the molecular oxygen-containing gas supplied to the reactor or the reactor temperature is controlled according to the oxygen concentration, thereby keeping the oxygen concentration in the product gas to a range of 2.5 to 8 vol %.

<7> The production process of a conjugated diene as described in any one of <1> to <6>, wherein the raw material gas is a gas containing 1-butene, cis-2-butene, trans-2-butene or a mixture thereof obtained by dimerization of ethylene, or a gas containing hydrocarbons having a carbon atom number of 4 obtained when fluid catalytically cracking a heavy oil fraction or a butene fraction produced by dehydrogenation or oxidative dehydrogenation reaction of n-butane.

### ADVANTAGE OF THE INVENTION

According to the present invention, in producing a conjugated diene by an oxidative dehydrogenation reaction of a monoolefin having a carbon atom number of 4 or more, a carbon portion can be prevented from accumulation such as coking on a catalyst in a reactor, the amount of high-boiling-point byproducts precipitated in a cooling step after the reaction step can be reduced, and a safer, continuous and stable operation of the plant can be realized.

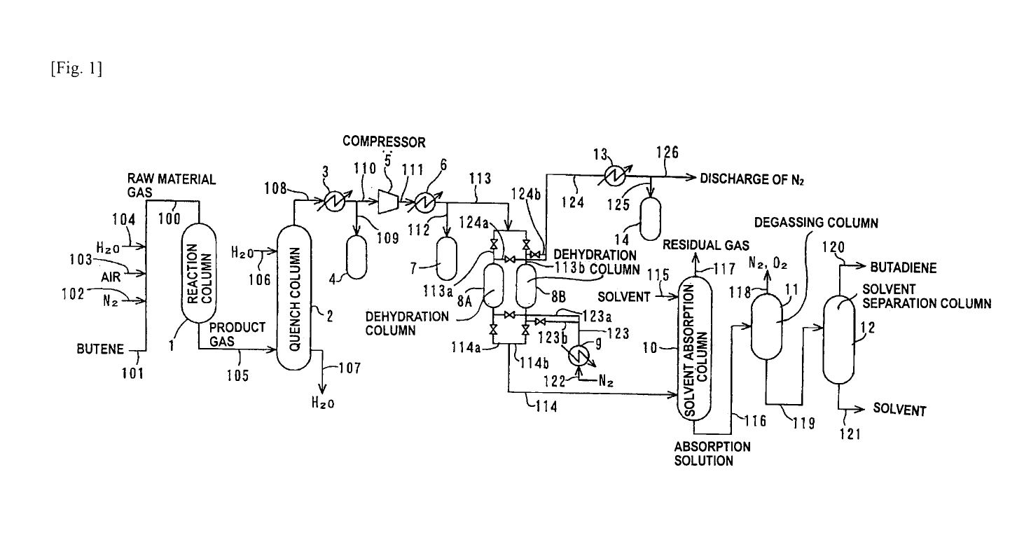

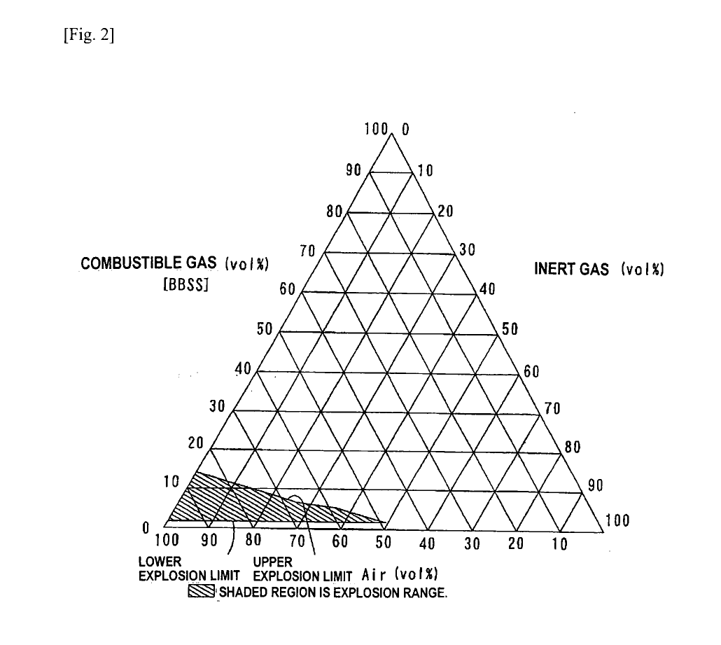

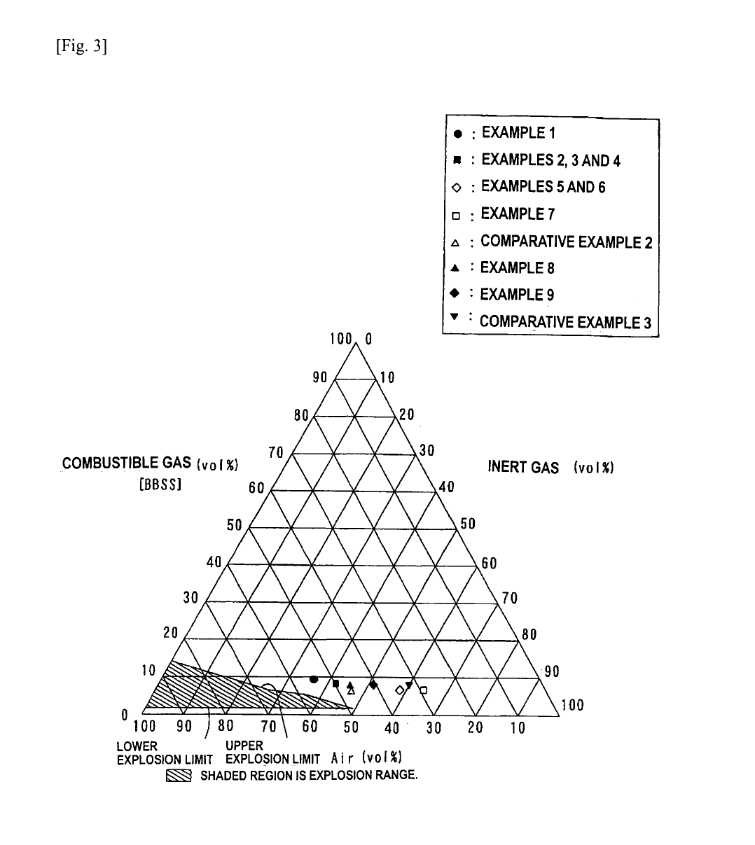

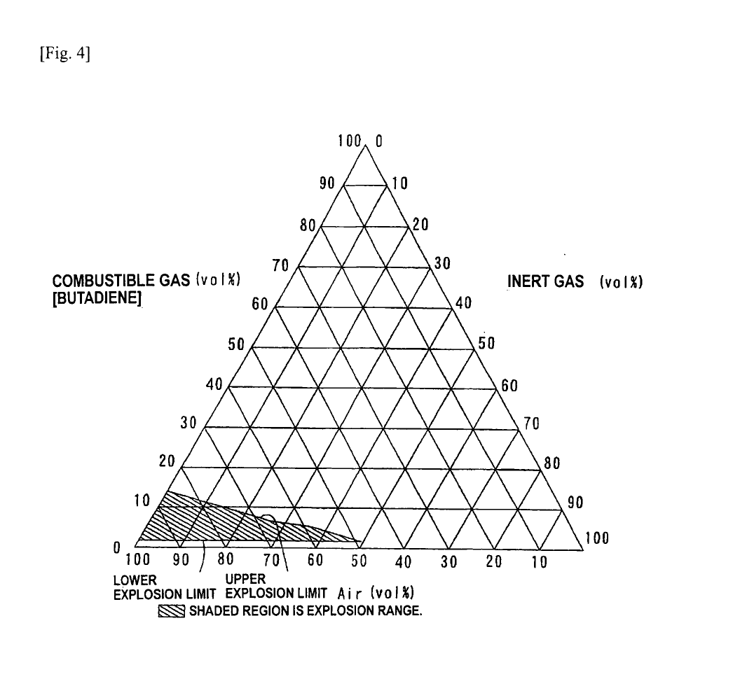

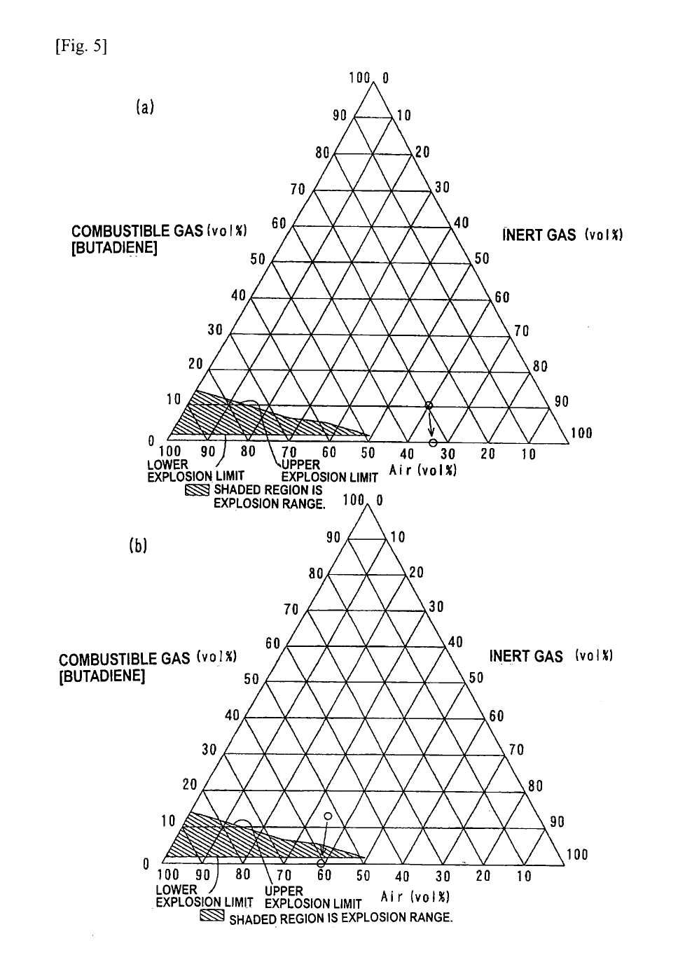

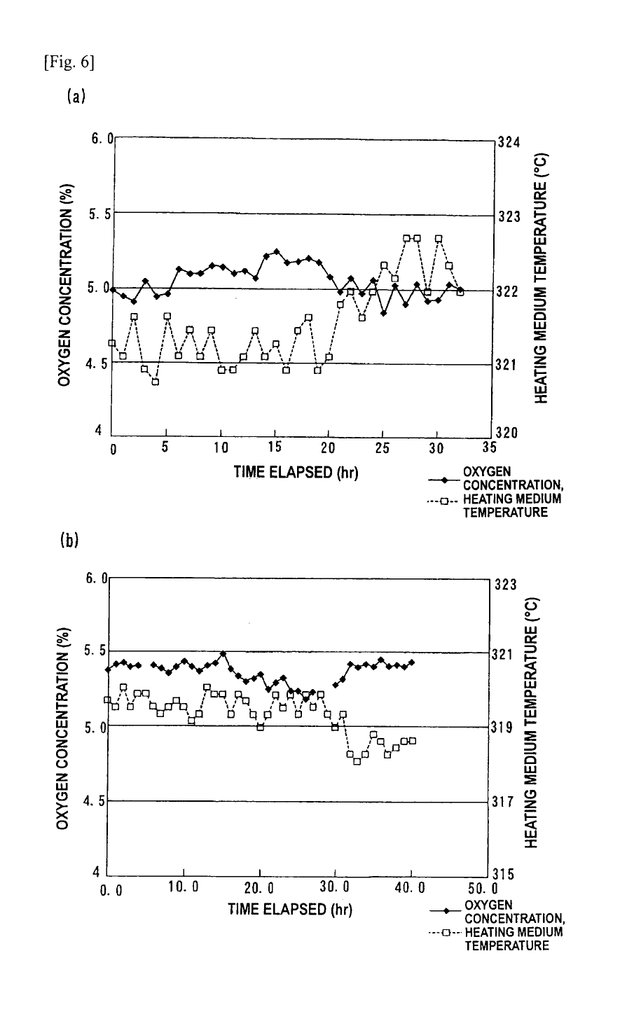

### BRIEF DESCRIPTION OF THE DRAWINGS

[FIG. 1] A process diagram showing the mode for carrying out the production process of a conjugated diene of the present invention.

[FIG. 2] A three-component diagram showing the explosion range of combustible gas (BBSS)-air-inert gas.

[FIG. 3] A three-component diagram showing the state of combustible gas concentration in the gas at the reactor inlet in Examples 1 to 9 and Comparative Examples 2 and 3.

[FIG. 4] A three-component diagram showing the explosion range of combustible gas (butadiene)-air-inert gas.

[FIG. 5] (a) A three-component diagram showing the change in the combustible gas concentration between before and after a solvent absorption column of the product gas in Example 1; and (b) a three-component diagram showing the change in the combustible gas concentration between before and after a solvent absorption column of the product gas in Comparative Example 1

[FIG. 6] (a) A graph showing the oxygen concentration at the outlet of a cooler 3 and the reactor heating medium temperature in Example 2; and (b) a graph showing the oxygen concentration at the outlet of a cooler 3 and the reactor heating medium temperature in Example 3.

[FIG. 7] A process diagram showing the extraction and separation process of butadiene from a C₄ fraction.

### MODE FOR CARRYING OUT THE INVENTION

The mode for carrying out the production process of a conjugated diene of the present invention is described in detail below, but the description in the following is one example (representative example) of the mode for carrying out the present invention, and the present invention is not limited to these contents.

In the present invention, a raw material gas containing a monoolefin having a carbon atom number of 4 or more and a molecular oxygen-containing gas are supplied to a reactor containing a catalytic layer, and a corresponding conjugated diene is produced by an oxidative dehydrogenation reaction.

### <Raw Material Gas Containing Monoolefin Having a Carbon Atom Number of 4 or More>

The raw material gas for use in the present invention contains a monoolefin having a carbon atom number of 4 or more, and the monoolefin having a carbon atom number of 4 or more includes a monoolefin having a carbon atom number of 4 or more, preferably a carbon atom number of 4 to 6, such as butene (e.g., n-butene such as 1-butene and/or 2-butene, isobutene), pentene, methylbutene and dimethylbutene, and can be effectively applied to the production of a corresponding conjugated diene by a catalytic oxidative dehydrogenation reaction. Above all, the present invention is most suitably used for the production of butadiene from n-butene (n-butene such as 1-butene and/or 2-butene).

As the raw material gas containing a monoolefin having a carbon atom number of 4 or more, it is not necessary to use an isolated monoolefin having a carbon atom number of 4 or more itself, and the gas may be used in an arbitrary mixture form, if desired. For example, in the case of obtaining butadiene, a high-purity n-butene (1-butene and/or 2-butene) may be used as the raw material gas, but a fraction (BBSS) comprising, as a main component, n-butene (1-butene and/or 2-butene) obtained by separating butadiene and i-butene (isobutene) from a C4 fraction (BB) by-produced in the above-described naphtha cracking, or a butene fraction produced by a dehydrogenation or oxidative dehydrogenation reaction of n-butane may be also used. Furthermore, a gas containing high-purity 1-butene, cis-2-butene, trans-2-butene or a mixture thereof obtained by dimerization of ethylene may be also used as the raw material gas. Incidentally, for the ethylene above, ethylene obtained by ethane dehydrogenation, ethanol dehydration, naphtha cracking or the like method may be used. In addition, a gas containing many hydrocarbons having a carbon atom number of 4 (hereinafter, this gas is sometimes simply referred to as FCC-C4) obtained by fluid catalytic cracking where a heavy oil fraction obtained when distilling crude oil in a petroleum refining plant or the like is decomposed using a powdered solid catalyst in a fluidized bed state and converted into a low-boiling-point hydrocarbon, may be directly used as the raw material gas, or a gas after removing impurities such as phosphorus and arsenic from FCC-C4 may be used as the raw material gas. The term “main component” as used herein indicates that the component accounts for usually 40 vol % or more, preferably 60 vol % or more, more preferably 75 vol % or more, still more preferably 99 vol % or more, based on the raw material gas.

The raw material gas for use in the present invention may contain arbitrary impurities within the range not inhibiting the effects of the present invention. In the case of producing butadiene from n-butene (1-butene and 2-butene), specific examples of the impurity which may be contained include a branched monoolefin such as isobutene; a saturated hydrocarbon such as propane, n-butane, i-butane and pentane; an olefin such as propylene and pentene, a diene such as 1,2-butadiene; and acetylenes such as methyl acetylene, vinyl acetylene and ethyl acetylene. The amount of the impurity is usually 40% or less, preferably 20% or less, more preferably 10% or less, still more preferably 1% or less. If this amount is too large, the concentration of 1-butene or 2-butene as the main raw material is decreased and this tends to slow the reaction or reduce the yield of butadiene that is the objective product. Also, in the present invention, the concentration of a linear monoolefin having a carbon atom number of 4 or more in the raw material gas is not particularly limited but is usually from 70.00 to 99.99 vol %, preferably from 71.00 to 99.0 vol %, more preferably from 72.00 to 95.0 vol %.

### <Oxidative Dehydrogenation Reaction Catalyst>

The oxidative dehydrogenation reaction catalyst suitably used in the present invention is described below. The oxidative dehydrogenation catalyst for use in the present invention is preferably a composite oxide catalyst containing at least molybdenum, bismuth and cobalt. Above all, the catalyst is preferably a composite oxide catalyst represented by the following formula (1):

MoₐBi_{b}Co_{c}Ni_{d}FeₑX_{f}Y_{g}ZₕSiᵢOⱼ (1)

In the formula, X is at least one element selected from the group consisting of magnesium (Mg), calcium (Ca), zinc (Zn), cerium (Ce) and samarium (Sm), Y is at least one element selected from the group consisting of sodium (Na), potassium (K), rubidium (Rb), cesium (Cs) and thallium (Tl), and Z is at least one element selected from the group consisting of boron (B), phosphorus (P), arsenic (As) and tungsten (W).

Furthermore, a to j represent an atomic ratio of respective elements and when a=12, are in ranges of b=0.5 to 7, c=0 to 10, d=0 to 10 (provided that c+d=1 to 10), e=0.05 to 3, f=0 to 2, g=0.04 to 2, h=0 to 3 and i=5 to 48, and j is a numerical value satisfying the oxidation state of other elements.

The composite oxide catalyst above is preferably produced through a step including integration in an aqueous system and heating of supply source compounds of respective component elements constituting the composite oxide catalyst. For example, all of supply source compounds of respective component elements may be integrated in an aqueous system and heated.

Above all, the composite oxide catalyst is preferably produced by a method comprising a pre-step of producing a catalyst precursor by heat-treating an aqueous solution or aqueous water dispersion of the raw material compound containing silica and at least one member selected from the group consisting of a molybdenum compound, an iron compound, a nickel compound and a cobalt compound, or a dry matter resulting from drying of the aqueous solution or aqueous water dispersion, and a post-step of integrating the catalyst precursor, a molybdenum compound and a bismuth compound together with an aqueous solvent, and drying and firing the mixture. When this method is used, the obtained composite oxide catalyst exerts high catalytic activity, so that a conjugated diene such as butadiene can be produced at a high yield and a reaction product gas with a small aldehyde content can be obtained. Incidentally, the aqueous solvent indicates water, an organic solvent having compatibility with water, such methanol and ethanol, or a mixture thereof.

The production method of a composite oxide catalyst suitable for the present invention is described below.

In the production method of this composite oxide catalyst, it is preferred that molybdenum used in the pre-step is molybdenum corresponding to a partial atomic proportion (a₁) out of the entire atomic proportion (a) of molybdenum and the molybdenum used in the post-step is molybdenum corresponding to the remaining atomic proportion (a₂) obtained by subtracting a₁ from the entire atomic proportion (a) of molybdenum. The a₁ is preferably a value satisfying 1<a₁/(c+d+e)<3, and the a₂ is preferably a value satisfying 0<a₂/b<8.

Examples of the supply source compound for the component element above include an oxide, a nitrate, a carbonate, an ammonium salt, a hydroxide, a carboxylate, an ammonium carboxylate, an ammonium halide, a hydroacid, an acetylacetonate and an alkoxide of the component element, and specific examples thereof include the followings.

Examples of the supply source compound for Mo include ammonium paramolybdate, molybdenum trioxide, molybdic acid, ammonium phosphomolybdate, and phosphomolybdic acid.

Examples of the supply source compound for Fe include ferric nitrate, ferric sulfate, ferric chloride, and ferric acetate.

Examples of the supply source compound for Co include cobalt nitrate, cobalt sulfate, cobalt chloride, cobalt carbonate, and cobalt acetate.

Examples of the supply source compound for Ni include nickel nitrate, nickel sulfate, nickel chloride, nickel carbonate, and nickel acetate.

Examples of the supply source compound for Si include silica, granular silica, colloidal silica, and fumed silica.

Examples of the supply source compound for Bi include bismuth chloride, bismuth nitrate, bismuth oxide, and bismuth subcarbonate. The compound may be also supplied as a composite carbonate compound of Bi and X component or Y component, where an X component (one element or two or more elements of Mg, Ca, Zn, Ce and Sm) or a Y component (one element or two or more elements of Na, K, Rb, Cs and Tl) is contained as a solid solution.

For example, in the case of using Na as the Y component, the composite carbonate compound of Bi and Na can be produced by adding dropwise and mixing an aqueous solution of a water-soluble bismuth compound such as bismuth nitrate in, for example, an aqueous solution of sodium carbonate or sodium bicarbonate, and washing and drying the obtained precipitate.

The composite carbonate compound of Bi and an X component can be produced by adding dropwise and mixing an aqueous solution composed of a water-soluble compound such as bismuth nitrate and nitrate of X component in, for example, an aqueous solution of ammonium carbonate or ammonium bicarbonate, and washing and drying the obtained precipitate.

When sodium carbonate or sodium bicarbonate is used instead of the ammonium carbonate or ammonium bicarbonate above, a composite carbonate compound of Bi, Na and X component can be produced.

Other examples of the supply source compound for the component element include the followings.

Examples of the supply source compound for K include potassium nitrate, potassium sulfate, potassium chloride, potassium carbonate, and potassium acetate.

Examples of the supply source compound for Rb include rubidium nitrate, rubidium sulfate, rubidium chloride, rubidium carbonate, and rubidium acetate.

Examples of the supply source compound for Cs include cesium nitrate, cesium sulfate, cesium chloride, cesium carbonate, and cesium acetate.

Examples of the supply source compound for Tl include thallous nitrate, thallous chloride, thallium carbonate, and thallous acetate.

Examples of the supply source compound for B include borax, ammonium borate, and boric acid.

Examples of the supply source compound for P include ammonium phosphomolybdate, ammonium phosphate, phosphoric acid, and phosphorus pentoxide.

Examples of the supply source compound for As include diarceno 18 ammonium molybdate, and diarceno 18 ammonium tungstate.

Examples of the supply source compound for W include ammonium paratungstate, tungsten trioxide, tungstic acid, and phosphotungstic acid.

Examples of the supply source compound for Mg include magnesium nitrate, magnesium sulfate, magnesium chloride, magnesium carbonate, and magnesium acetate.

Examples of the supply source compound for Ca include calcium nitrate, calcium sulfate, calcium chloride, calcium carbonate, and calcium acetate.

Examples of the supply source compound for Zn include zinc nitrate, zinc sulfate, zinc chloride, zinc carbonate, and zinc acetate.

Examples of the supply source compound for Ce include cerium nitrate, cerium sulfate, cerium chloride, cerium carbonate, and cerium acetate.

Examples of the supply source compound for Sm include samarium nitrate, samarium sulfate, samarium chloride, samarium carbonate, and samarium acetate.

The aqueous solution or aqueous water dispersion of the raw material compound, which is used in the pre-step, is an aqueous solution, water slurry or cake containing, as catalyst components, at least molybdenum (corresponding a₁ out of the entire atomic proportion a), iron, at least either nickel or cobalt, and silica.

The aqueous solution or aqueous water dispersion of the raw material compound is prepared by integration of supply source compounds in an aqueous system. Here, the integration of supply source compounds of respective component elements in an aqueous system means that aqueous solutions or aqueous water dispersions of supply source compounds of respective component elements are at least either mixed or ripened en bloc or stepwise. That is, all of (a) a method of mixing respective supply source compounds en bloc, (b) a method of mixing respective supply source compounds en bloc and ripening the mixture, (c) a method of stepwise mixing respective supply source compounds, (d) a method of repeating stepwise mixing•ripening of respective supply source compounds, and a combination of (a) to (d) are included in the concept of integration of supply source compounds of respective component elements in an aqueous system. Here, the ripening indicates an operation of treating the industrial raw material or half-finished product under specific conditions such as given time and given temperature with an attempt to acquire or raise the required physical properties or chemical properties or allow the progress of a predetermined reaction. The given time is usually from 10 minutes to 24 hours, and the given temperature is usually from room temperature to the boiling point of the aqueous solution or aqueous water dispersion.

The specific method for integration includes, for example, a method where a solution obtained by mixing acidic salts selected from the catalytic components and a solution obtained by mixing basic salts selected from the catalytic components are mixed, and specific examples thereof include a method of adding, under heating, a mixture containing an iron compound and at least either a nickel compound or cobalt compound to an aqueous solution of molybdenum compound, and mixing silica therewith.

The thus-obtained aqueous solution or aqueous water dispersion of the raw material compound containing silica is heated at 60 to 90° C. and ripened.

The ripening means to stir the slurry for catalyst precursor at a predetermined temperature for a predetermined time. By this ripening, the viscosity of the slurry is raised, sedimentation of a solid component in the slurry is slowed, and this is effective particularly in preventing disproportionation of components in the next drying step, as a result, the catalytic activity such as raw material conversion and selectivity of the composite oxide catalyst as the final product is more improved.

The temperature at the ripening is preferably from 60 to 90° C., more preferably from 70 to 85° C. If the ripening temperature is less than 60° C., the effect of ripening is insufficient and good activity may not be obtained, whereas if it exceeds 90° C., much water evaporates during the ripening time and this is disadvantageous in industrial practice. Furthermore, if the ripening temperature exceeds 100° C., a pressure-resistant vessel is required for the dissolution tank or handling becomes cumbersome, and this is significantly disadvantageous in view of profitability and operability.

The time for which the ripening is applied is suitably from 2 to 12 hours, preferably from 3 to 8 hours. If the ripening time is less than 2 hours, the activity and selectivity of the catalyst may not be fully brought out, whereas even if it exceeds 12 hours, the ripening effect is not increased and this is disadvantageous in industrial practice.

As the stirring method, an arbitrary method can be employed, and examples thereof include a method by a stirrer having a stirring blade, and a method by external circulation using a pump.

The ripened slurry is heat-treated directly or after drying. In the case of drying the slurry, the drying method and the condition of the obtained dry matter are not particularly limited, and, for example, a powdered dry matter may be obtained using a normal spray drier, slurry drier, drum drier or the like, or a block-like or flake-like dry matter may be obtained using a normal box-type drier or tunnel-type firing furnace.

The aqueous solution of raw material salts or a granule or cake obtained by drying the solution is heat-treated in air in a temperature region of 200 to 400° C., preferably from 250 to 350° C., for a short time. At this time, the form of the furnace and the method therefor are not particularly limited, and, for example, heating may be performed using a normal box-type heating furnace or tunnel-type heating furnace in a state of the dry matter being fixed. Also, heating may be performed using a rotary kiln or the like while fluidizing the dry matter.

The ignition loss of the catalyst precursor obtained after heat treatment is preferably from 0.5 to 5 wt %, more preferably from 1 to 3 wt %. By adjusting the ignition loss to this range, a catalyst having a high raw material conversion or a high selectivity can be obtained. Incidentally, the ignition loss is a value obtained according to the following formula:

Ignition loss (%)=[(W₀−W₁)/W₀]×100

W₀: Weight (g) after the catalyst precursor is dried at 150° C. for 3 hours to remove adhering moisture.

W₁: Weight (g) after the catalyst precursor deprived of adhering moisture is further heat-treated at 500° C. for 2 hours.

In the post-step, integration of the catalyst precursor obtained in the pre-step, a molybdenum compound (a₂ remaining after subtracting a₁ from the entire atomic proportion a), and a bismuth compound is performed in an aqueous solvent. At this time, it is preferred to add aqueous ammonia. Addition of X, Y and Z components is also preferably performed in this post-step. The bismuth supply source compound for use in the present invention is a sparingly water-soluble or water-insoluble bismuth. This compound is preferably used in a powder form. These compounds as raw materials for the catalyst production may be a particle larger than a powder, but considering a heating step of which heat should be diffused, a smaller particle is preferred. Accordingly, when the compounds as raw materials are not such a small particle, pulverization should be performed before the heating step.

The obtained slurry is then thoroughly stirred and dried. The dry product obtained in this way is molded into an arbitrary shape by a method such as extrusion molding, tablet molding or carrier molding.

The shaped product is then preferably subjected to a final heat treatment under the temperature condition of 450 to 650° C. for approximately from 1 to 16 hours. In this way, a composite oxide catalyst having high activity and giving the objective oxidation product at a high yield is obtained.

### <Molecular Oxygen-Containing Gas>

The molecular oxygen-containing gas for use in the present invention is a gas containing molecular oxygen in an amount of usually 10 vol % or more, preferably 15 vol % or more, more preferably 20 vol % or more, and specifically, air is preferred. Also, in view of increase in the cost necessary for industrially preparing a molecular oxygen-containing gas, the upper limit of the molecular oxygen content is usually 50 vol % or less, preferably 30 vol % or less, more preferably 25 vol % or less. Furthermore, the molecular oxygen-containing gas may contain arbitrary impurities within the range not impairing the effects of the present invention.

Specific examples of the impurity which may be contained include nitrogen, argon, neon, helium, CO, CO₂ and water. The amount of the impurity is, in the case of nitrogen, usually 90 vol % or less, preferably 85 vol % or less, more preferably 80 vol % or less. In the case of a component other than nitrogen, the amount is usually 10 vol % or less, preferably 1 vol % or less. If this amount is too large, supplying oxygen necessary for the reaction tends to become difficult.

### <Gas Supply>

In the present invention, at the time of supplying the raw material gas to the reactor, it is necessary to mix the raw material gas with the molecular oxygen-containing gas and supply the gas after mixing (hereinafter, sometimes referred to as a “mixed gas”) to the reactor. In the mixed gas for use in the present invention, the proportion of the raw material gas is usually 4.2 vol % or more, preferably 7.6 vol % or more, more preferably 8.0 vol % or more. There is a tendency that when this lower limit value becomes larger, the reactor size can be smaller and the cost involved in construction and operation is reduced. On the other hand, the upper limit is 20.0 vol % or less, preferably 17.0 vol % or less, more preferably 15.0 vol % or less. As the upper limit value becomes smaller, the content of a substance giving rise to coking on the catalyst in the raw material gas is also reduced and coking of the catalyst is advantageously less likely to occur.

### <Nitrogen Gas, Water (Steam)>

Together with the mixed gas, a nitrogen gas and water (steam) may be also supplied to the reactor. A nitrogen gas is added for adjusting the concentrations of combustible gas and oxygen so as not to allow the mixed gas to form a detonating gas, and water (steam) is added for adjusting the concentrations of combustible gas and oxygen, similarly to the nitrogen gas, as well as for suppressing coking of the catalyst. For these reasons, it is preferred to further mix water (steam) and a nitrogen gas with the mixed gas and supply the resulting gas to the reactor.

In the case of supplying steam to the reactor, the steam is preferably introduced in a ratio of 0.5 to 5.0 based on the supplied amount of the raw material gas. As this ratio becomes larger, the amount of wastewater tends to increase, and as it becomes smaller, the yield of the objective product butadiene is liable to decrease. For these reasons, steam is introduced in a ratio of preferably from 0.8 to 4.5, more preferably from 1.0 to 4.0, based on the supplied amount of the raw material gas.

In the case of supplying a nitrogen gas to the reactor, the nitrogen gas is preferably introduced in a ratio (volume ratio) of 0.5 to 8.0 based on the supplied amount of the raw material gas. As this ratio becomes larger, the load imposed on the step of compressing the product gas in the post-step tends to rise, and as it becomes smaller, the amount used of steam supplied to the reactor is liable to increase. For these reasons, the nitrogen gas is introduced in a ratio of preferably from 1.0 to 6.0, more preferably from 2.0 to 5.0, based on the supplied amount of the raw material gas.

The method for supplying the mixed gas of the raw material gas and the molecular oxygen-containing gas and supplying a nitrogen gas and water (steam) which are supplied, if desired, is not particularly limited, and these may be supplied through separate pipings but in order to unfailingly avoid formation of a detonating gas, the mixed gas is preferably supplied after previously supplying a nitrogen gas to the raw material gas or molecular oxygen-containing gas before obtaining the mixed gas, and in this state, mixing the raw material gas and the molecular oxygen-containing gas to obtain the mixed gas.

A representative composition of the mixed gas is illustrated below.

### [Mixed Gas Composition]

n-butene: from 50 to 100 vol % based on the total of C₄ fractions

Total of C₄ fractions: from 5 to 15 vol %

O₂: from 40 to 120 vol/vol % based on the total of C₄ fractions

N₂: from 500 to 1,000 vol/vol % based on the total of C₄ fractions

H₂O: from 90 to 900 vol/vol % based on the total of C₄ fractions

The mixed gas supplied to the reactor is a mixture of oxygen and a combustible gas and therefore, the composition of the mixed gas at the reactor inlet is controlled while monitoring the flow rate by a flowmeter disposed in piping for supplying each of the gases (raw material gas, air, and, if desired, nitrogen gas and water (steam)) to keep apart from the explosion range, whereby the mixed gas composition can be adjusted to the composition described above (in the case of using C₄ fractions).

The “explosion range” as used herein means a range where the gas containing oxygen and a combustible gas has a composition allowing ignition in the presence of some ignition source. For example, in the case of using BBSS as a combustible gas and using this gas, air and an inert gas (N₂ gas), as a result of measurement by the later-described method, the explosion range is the shaded area in the left lower part in the three-component diagram of combustible gas (BBSS)-air-inert gas shown in FIG. 2, and in the case of using 1,3-butadiene as a combustible gas and using this gas, air and an inert gas (N₂ gas), as a result of measurement by the later-described method, the explosion range is the shaded area in the left lower part in the three-component diagram of combustible gas-air-inert gas shown in FIG. 4.

It is generally known that when the combustible gas concentration in the gas is lower than a certain value, ignition does not occur even in the presence of an ignition source, and this concentration is referred to as a lower explosion limit. Also, it is known that when the combustible gas concentration in the gas is higher than a certain value, ignition does not occur even in the presence of an ignition source, and this concentration is referred to as an upper explosion limit. Each value depends on the oxygen concentration in the gas. In general, as the oxygen concentration is lower, both values come close to each other and when the oxygen concentration reaches a certain value, both agree. The oxygen concentration here is referred to as a threshold oxygen concentration and when the oxygen concentration is lower than that, gas is not ignited irrespective of the combustible gas concentration.

In the present invention, the combustible gas concentration in the gas supplied to an oxidative dehydrogenation reactor must be higher than the upper explosion limit, and it is preferred that at the time of starting an oxidative dehydrogenation reaction, supply of the combustible gas (mainly, the raw material gas) is started after previously adjusting the oxygen concentration in the mixed gas at the reactor inlet to lower than the threshold oxygen concentration by controlling the amounts of the molecular oxygen-containing gas, nitrogen and steam supplied to the reactor, and thereafter, the supplied amounts of the combustible gas (mainly, the raw material gas) and the molecular oxygen-containing gas such as air are increased to raise the combustible gas concentration in the mixed gas to higher than the upper explosion limit.

In the course of increasing the supplied amount of the combustible gas (mainly, the raw material gas) and the molecular oxygen-containing gas, the supplied amount of at least either one of nitrogen and steam may be decreased to keep the supplied amount of the mixed gas constant. This makes it possible to keep a constant residence time of the mixed gas in piping and reactor and suppress fluctuation of the pressure.

In the present invention, a mixed gas having a combustible gas concentration not less than the upper explosion limit is supplied to the reactor and an oxidative dehydrogenation reaction is performed in the presence of a catalyst to obtain a product gas, but when the combustible gas in the mixed gas composition at the reactor inlet is not less than the explosion limit, the combustible gas concentration is kept from reduction due to the oxidative dehydrogenation reaction. Therefore, the composition at the reactor outlet is usually also not less than the upper explosion limit, and there is not danger of an explosion.

In the present invention, in the case of including a step of contacting the later-described product gas with an absorption solvent to let hydrocarbons such as olefin and conjugated diene be absorbed by the absorption solvent and thereby obtain a conjugated diene-containing solvent (hereinafter, sometimes referred to as a “solvent absorption step”), in the solvent absorption step, the concentration of the combustible gas such as hydrocarbon in the product gas may decrease and fall in the explosion range. For avoiding this, it may be considered to contact the product gas with an absorption solvent after diluting it with an inert gas such as nitrogen, but an easy and simple method is to let the composition at the rector outlet be not more than the threshold oxygen concentration by previously adjusting the reaction conditions.

Furthermore, in the present invention, the oxygen concentration in the product gas must be 8.0 vol % or less and is preferably 7.5 vol % or less, more preferably 7.0 vol % or less. As this upper limit value becomes smaller, even when the combustible gas such as conjugated diene is absorbed by a solvent in the solvent absorption step, the gas composition can be more prevented from falling in the explosion range and moreover, the content of a byproduct solid matter in the product gas tends to decrease. On the other hand, the oxygen concentration in the product gas must be 2.5 vol % or more and is preferably 3 vol % or more, more preferably 4.0 vol % or more. As this lower limit value becomes larger, attachment of a carbon portion or the like to the catalyst surface (coking) can be more reduced.

The oxygen concentration in the product gas can be measured at the reactor outlet or in the post-step of the reaction by using a known oximeter such as magnetic dumbbell system, or a gas chromatography.

In order to maintain the oxygen concentration in the product gas in the range of 2.5 to 8.0 vol %, at least either one of the amount of oxygen supplied to the reactor and the reactor temperature is preferably manipulated according to the oxygen concentration in the product gas measured. Specifically, for example, when the target oxygen concentration is set in an oxygen concentration range of 2.5 to 8.0 vol % and the oxygen concentration is lower than the target range, the oxygen concentration at the reactor outlet is raised by increasing the flow rate of oxygen supplied to the reactor, lowering the temperature of the reactor, or executing both, and when the oxygen concentration is higher than the target concentration, the oxygen concentration at the reactor outlet is reduced by decreasing the flow rate of oxygen supplied to the reactor, raising the temperature of the reactor, or executing both. By these manipulations, the oxygen concentration in the product gas measured between the reactor 1 outlet and the solvent absorption column 10 can be maintained in the range of 2.5 to 8.0 vol %.

Incidentally, if the supplied oxygen amount is too small, the lattice oxygen of the oxidative dehydrogenation catalyst is consumed by the reaction to cause collapse of the crystal structure and the reaction catalyst may be deteriorated. For this reason, oxygen is preferably supplied to the reactor so that the oxygen concentration in the product gas can be 2.5 vol % or more. Also, in order to keep the oxygen concentration in the product gas from exceeding 8.0 vol %, the product gas may be diluted with an inert gas such as nitrogen so as to reduce the oxygen concentration to 8.0 vol % or less, but it is economically disadvantageous to daringly add an inert gas or the like component which should be separated in the solvent absorption step.

### <Reactor>

The reactor used for the oxidative dehydrogenation reaction of the present invention is not particularly limited but, specifically, includes a tube-type reactor, a tank-type reactor and a fluidized bed reactor. A fixed-bed reactor is preferred, a fixed-bed multitubular reactor or a plate-type reactor is more preferred, and a fixed-bed multitubular reactor is most preferred.

In the case where the reactor is a fixed-bed reactor, a catalytic layer having the above-described oxidative dehydrogenation reaction catalyst is present in the reactor. The catalytic layer may consist of a layer composed of only a catalyst, may consist of only a layer containing a catalyst and a solid matter nonreactive with the catalyst, or may consist of a plurality of layers, that is, a layer containing a catalyst and a solid matter nonreactive with the catalyst and a layer composed of only a catalyst. When the catalytic layer comprises a layer containing a catalyst and a solid matter nonreactive with the catalyst, the catalytic layer can be kept from an abrupt temperature rise due to heat generation during reaction. In the case of having a plurality of layers, the plurality of layers are formed in a stratified manner in the direction from the reactor inlet toward the product gas exit of the reactor. In the case where the catalytic layer comprises a layer containing a catalyst and a solid matter nonreactive with the catalyst, the catalyst dilution ratio represented by the following formula is preferably 10 vol % or more, more preferably 20 vol % or more, still more preferably 30 vol % or more. As this lower limit value becomes larger, generation of a hot spot in the catalytic layer can be more suppressed and the effect of preventing accumulation of a carbon portion on the catalyst is increased. The upper limit of the dilution ratio of the catalytic layer is not particularly limited but is usually 99 vol % or less, preferably 90 vol % or less, more preferably 80 vol % or less. As this upper limit value becomes smaller, the reactor size can be smaller and the cost involved in construction and operation can be reduced.

As described above, the catalytic layer provided in the reactor may be a single layer or two or more layers but is preferably from 2 to 5 layers, more preferably from 3 to 4 layers. As the number of catalytic layers is larger, the catalyst packing operation tends to become more cumbersome, and as the number of catalytic layers is smaller, the operation is liable to be easier. In the case of two or more catalytic layers in the reactor, the dilution ratio of each catalytic layer may be appropriately determined according to reaction conditions or reaction temperature, but it is preferred to provide catalytic layers differing in the dilution ratio.

Dilution ratio (vol %)=[(volume of solid matter nonreactive with catalyst)/(volume of catalyst+volume of solid matter nonreactive with catalyst)]×100

The nonreactive solid matter for use in the present invention is not particularly limited as long as it is stable under the reaction conditions for production of a conjugated diene and nonreactive with the raw material substance such as monoolefin having a carbon atom number of 4 or more and the product such as conjugated diene, and this may be generally called an inert ball. Specific examples thereof include a ceramic material such as alumina and zirconia. Also, the shape thereof is not particularly limited and may be any of sphere, column, ring and amorphous. The size thereof may be sufficient if it is equal to that of the catalyst used in the present invention. The particle size of the solid matter is usually on the order of 2 to 10 mm.

The packed length of the catalytic layer can be determined by calculations of material balance and heat balance when the activity of catalyst packed (in the case of being diluted with a nonreactive solid matter, the activity as the diluted catalyst), the size of reactor, the temperature of reaction raw material gas, the reaction temperature and the reaction conditions are decided.

### <Reaction Conditions>

The oxidative dehydrogenation reaction in the present invention is an exothermic reaction and the temperature rises by the reaction, but in the present invention, the reaction temperature is usually adjusted to be from 250 to 450° C., preferably from 280 to 400° C. As this temperature becomes higher, the catalytic activity tends to be rapidly reduced, and as it becomes lower, the yield of the conjugated diene that is the objective product is liable to decrease. The reaction temperature can be controlled using a heating medium (e.g., dibenzyltoluene, nitrite). The reaction temperature as used herein indicates the temperature of the heating medium.

The temperature in the reactor for use in the present invention is not particularly limited but is usually from 250 to 450° C., preferably from 280 to 400° C., more preferably from 320 to 395° C. If the temperature of the catalytic layer exceeds 450° C., this involves a tendency that as the reaction continues, the catalytic activity may be rapidly reduced, whereas if the temperature of the catalytic layer is less than 250° C., the yield of the conjugated diene that is the objective produce tends to be decreased. The temperature in the reactor is determined according to the reaction conditions and may be controlled, for example, by the dilution ratio of catalytic layer or the flow rate of mixed gas. The term “temperature in the reactor” as used herein indicates the temperature of the product gas at the reactor outlet and in the case of a reactor having a catalytic layer, indicates the temperature of the catalytic layer.

The pressure in the reactor used in the present invention is not particularly limited, but the lower limit is usually 0 MPaG or more, preferably 0.001 MPaG or more, more preferably 0.01 MPaG or more. As this value becomes larger, a larger amount of the reaction gas can be advantageously supplied to the reactor. On the other hand, the upper limit is 0.5 MPaG or less, preferably 0.3 MPaG or less, more preferably 0.1 MPaG or less. As this value becomes smaller, the explosion range tends to be narrower.

The residence time in the reactor used in the present invention is not particularly limited, but the lower limit is usually 0.36 seconds or more, preferably 0.80 seconds or more, more preferably 0.90 seconds or more. As this value becomes larger, the conversion of the monoolefin in the raw material gas is advantageously increased. On the other hand, the upper limit is 3.60 seconds or less, preferably 2.80 seconds or less, more preferably 2.10 seconds or less. As this value becomes smaller, the size of the reactor tends to be reduced.

Also, in the present invention, the ratio of the flow rate of the mixed gas to the amount of the catalyst in the reactor is from 1,000 to 10,000 h⁻¹, preferably from 1,300 to 4,500 h⁻¹, more preferably 1,700 to 4,000 h⁻¹. As this value becomes larger, precipitation of a solid matter tends to be suppressed, and as it becomes smaller, a solid matter is liable to be precipitated.

The difference in the flow rate between the inlet and the outlet of the reactor depends on the flow rate of the raw material gas at the reactor inlet and the flow rate of the product gas at the reactor outlet, but the ratio of the flow rate at the outlet to the flow rate at the inlet is usually from 100 to 110 vol %, preferably from 102 to 107 vol %, more preferably from 103 to 105 vol %. In the case of producing butadiene from n-butene (1-butene and 2-butene), the flow rate at the outlet increases, because the number of molecules is stoichiometrically increased by the reaction of producing butadiene and water resulting from oxidative dehydrogenation of butene or the reaction of producing CO or CO₂ in a side reaction. A small increase in the flow rate at the outlet disadvantageously indicates that the reaction is not proceeding, and an excessive increase in the flow rate at the outlet is not preferred, because the amount of CO or CO₂ produced by a side reaction is increased.

Thus, by the oxidative dehydrogenation reaction of a monoolefin in the raw material gas, a conjugated diene corresponding to the monoolefin is produced, and a product gas containing the conjugated diene is obtained. The concentration of the conjugated diene contained in the product gas, which corresponds to the monoolefin in the raw material gas, depends on the concentration of the monoolefin contained in the raw material gas but is usually from 1 to 15 vol %, preferably from 5 to 13 vol %, more preferably from 9 to 11 vol %. A larger conjugated diene concentration is advantageous in that the recovery cost is low, and a smaller concentration is advantageous in that a side reaction such as polymerization is less likely to occur when the product gas is compressed in the next step. In the product gas, an unreacted monoolefin may be contained, and the concentration thereof is usually from 0 to 7 vol %, preferably from 0 to 4 vol %, more preferably from 0 to 2 vol %. Incidentally, in the present invention, the high-boiling-point byproduct contained in the product gas varies depending on the kind of the impurity contained in the raw material gas used but indicates a byproduct having a boiling point of 200 to 500° C. under atmospheric pressure. In the case of producing butadiene from n-butene (1-butene and 2-butene), specific examples of the high-boiling-point byproduct include phthalic acid, anthraquinone and fluorenone. The amount thereof is not particularly limited but is usually from 0.05 to 0.10 vol % based on the reaction gas.

### <Post-Step>

The production process of a conjugated diene of the present invention may further include a cooling step, a dehydration step, a solvent absorption step, a separation step, a purification step and the like so as to separate a conjugated diene from the conjugated diene-containing product gas. Incidentally, the product gas obtained from the reactor turns into a compressed gas and a dehydrated gas in the dehydration step. However, these gases contain the components in the same ratio except for water and since most of water contained is in a liquid state, the ratio of components in the gas portion may be considered to be the same between respective gases. For this reason, in the following, the product gas, the compressed gas and the dehydrated gas are sometimes simply referred to as a “product gas”.

### (Cooling Step)

In the present invention, a cooling step of cooling the conjugated diene-containing product gas obtained from the reactor may be provided. The cooling step is not particularly limited as long as it is a step capable of cooling the product gas obtained from the reactor outlet, but a method of brining a cooled solvent into direct contact with the product gas, thereby cooling the gas, is suitably used. The cooled solvent is not particularly limited but is preferably water or an aqueous alkali solution and most preferably water.

The cooling temperature of the product gas varies depending on the temperature of the product gas obtained from the reactor outlet, the kind of the cooled solvent, and the like, but the product gas is cooled to usually from 5 to 100° C., preferably from 10 to 50° C., more preferably from 15 to 40° C. As the temperature to which the product gas is cooled is higher, the cost involved in construction and operation tends to be reduced, and as the temperature is lower, the load imposed on the step of compressing the product gas is liable to be relieved. The pressure in the cooling column is not particularly limited but is usually 0.03 MPaG. When many high-boiling-point byproducts are contained in the product gas, polymerization of high-boiling-point byproducts with each other or deposition of a solid precipitate attributable to the high-boiling-point byproduct in the step is liable to occur. Also, the cooled solvent used in the cooling column is often circulated for utilization and therefore, when the production of a conjugated diene is uninterruptedly continued, clogging due to a solid precipitate may occur.

For this reason, high-boiling-point byproducts in the product gas are preferably not carried over into the cooling step as much as possible.

### (Dehydration Step)

In the present invention, a dehydration step of removing moisture contained in the product gas discharged from the reactor may be provided. By providing the dehydration step, corrosion of the equipment due to moisture in each step in the later process or accumulation of impurities on the solvent used in the later-described solvent absorption step or solvent separation step can be advantageously prevented.

The dehydration step in the present invention is not particularly limited as long as it is a step capable of removing moisture contained in the product gas. The dehydration step may be performed at any stage in the latter part of the reactor, but the dehydration step is preferably performed after the above-described cooling step. The amount of water contained in the product gas discharged from the reactor generally varies depending on, for example, the kind of raw material gas, the amount of molecular oxygen-containing gas and further, the steam mixed together with the raw material gas, but water is contained in an amount of usually from 4 to 35 vol %, preferably from 10 to 30 vol % (in the case of passing through a cooling step using water, the amount of water is reduced to a water concentration of 100 vol ppm to 2.0 vol %). The dew point is from 0 to 100° C., preferably 10 to 80° C.

The means for dehydrating water from the product gas is not particularly limited, but a desiccant (water adsorbent) such as calcium oxide, calcium chloride and molecular sieve may be utilized. Above all, in view of easy regeneration and easy handling, a desiccant (water adsorbent) such as molecular sieve is preferably utilized.

In the case of utilizing a desiccant such as molecular sieve in the dehydration step, other than water, high-boiling-point byproducts contained in the product gas are also removed by adsorption. High-boiling-point byproducts removed here are anthraquinone, fluorenone, phthalic acid and the like.

The water content in the product gas obtained through the dehydration step is usually from 10 to 10,000 vol ppm, preferably from 20 to 1,000 vol ppm, and the dew point is from −60 to 80° C., preferably from −50 to 20° C. As the water content in the product gas becomes larger, contamination of a reboiler in the solvent absorption column or solvent separation column tends to increase, whereas if it becomes smaller, the cost of utilities used in the dehydration step is liable to rise.

### (Solvent Absorption Step)

The present invention preferably includes a solvent absorption step of contacting the product gas with an absorption solvent to let hydrocarbons such as olefin and conjugated diene be absorbed by the absorption solvent and obtain a conjugated diene-containing solvent. As the reason why this step is preferred, from the standpoint of reducing the energy cost required for the separation of conjugated diene, the conjugated diene is preferably recovered by letting the product gas be absorbed by a solvent. The solvent absorption step may be performed at any stage in the latter part of the reactor but is preferably provided after the above-described dehydration step.

Specifically, the method for letting the product gas be absorbed by the solvent in the solvent absorption step is preferably, for example, a method using an absorption column. As for the kind of the absorption column, a packed column, a wet wall column, a spray column, a cyclone scrubber, a bubble column, a bubble-stirred tank, a tray column (bubble cap column, seive tray column), a foam separation column and the like can be used. A spray column, a bubble cap column and a seive tray column are preferred.

In the case of using an absorption column, a conjugated diene, an unreacted monoolefin having a carbon atom number of 4 or more, and a hydrocarbon compound having a carbon atom number of 3 or less, which are contained in the product gas, are absorbed by a solvent. Examples of the hydrocarbon compound having a carbon atom number of 3 or less include methane, acetylene, ethylene, ethane, methyl acetylene, propylene, propane and allene.

In the case where the product gas is recovered using an absorption column in the solvent absorption step, the pressure in the absorption column is not particularly limited but is usually from 0.1 to 2.0 MPaG, preferably from 0.2 to 1.5 MPaG, more preferably from 0.25 to 1.0 MPaG. As this pressure is higher, the absorption efficiency is advantageously more improved, and as the pressure is lower, there is an advantage that the energy required for raising the pressure at the time of introducing a gas into the absorption column can be more reduced and furthermore, the amount of dissolved oxygen in the liquid can be more reduced.

The temperature in the absorption column 10 is not particularly limited but is usually from 0 to 50° C., preferably from 10 to 40° C., more preferably from 20 to 30° C. As this temperature is higher, oxygen, nitrogen and the like are advantageously less likely to be absorbed into the solvent, and as the temperature is lower, there is an advantage that the absorption efficiency for a hydrocarbon such as conjugated diene is more improved.

The absorption solvent used in the solvent absorption step of the present invention is not particularly limited, but, for example, a saturated C₆-C₁₀ hydrocarbon, an aromatic C₆-C₈ hydrocarbon, and an amide compound are used. Specific examples of the solvent which can be used include dimethylformamide (DMF), toluene, xylene, and N-methyl-2-pyrrolidone (NMP). Among these, an aromatic C₆-C₈ hydrocarbon scarcely dissolves an inorganic gas and is preferred, and toluene is more preferred.

The amount of the absorption solvent used is not particularly limited but is usually from 1 to 100 times by weight, preferably from 2 to 50 times by weight, based on the flow rate of the objective product supplied to a recovery step. A larger amount of the absorption solvent used tends to be unprofitable, and a smaller amount is liable to cause reduction in the recovery efficiency of the conjugated diene.

In the conjugated diene-containing solvent obtained in the solvent absorption step, a conjugated diene that is the objective product is mainly contained, and the concentration of the conjugated diene in the solvent absorption liquid is usually from 1 to 20 wt %, preferably from 3 to 10 wt %. As the conjugated diene concentration in the solvent is higher, the loss of the conjugated diene due to polymerization or volatilization tends to increase, and as the concentration is lower, there is a tendency that the solvent amount required for circulation to give the same production amount increases and in turn, the energy cost necessary for the operation rises.

A slight amount of nitrogen or oxygen is also absorbed by the obtained conjugated diene-containing solvent and therefore, a degassing step of gasifying and thereby removing nitrogen or oxygen dissolved in the solvent may be provided. The degassing step is not particularly limited as long as it is a step capable of gasifying and thereby removing nitrogen or oxygen dissolved in the solvent absorption liquid.

### (Separation Step)

A separation step of separating a crude conjugated diene from the thus-obtained conjugated diene-containing solvent may be provided, and by this step, a crude conjugated diene can be obtained. The separation step is not particularly limited as long as it is a step capable of separating a crude conjugated diene from the solvent absorption liquid containing a conjugated diene, but the crude conjugated diene can be usually separated by distillation/separation. Specifically, for example, distillation/separation of the conjugated diene is performed by a reboiler and a condenser, and a conjugated diene fraction is withdrawn near the top. The separated absorption solvent is withdrawn from the bottom and in the case of having a recovery step of using the solvent in a step of the former stage, the solvent is circulated for utilization as an absorption solvent in the recovery step. Impurities may accumulate in the solvent during circulation for utilization, and it is preferred to extract a part and remove the impurities by a known purification method such as distillation, decantation, sedimentation and contact treatment with adsorbent or ion-exchange resin.

The pressure at distillation of a distillation column used in the separation step may be arbitrarily set, but usually, the top pressure is preferably set to from 0.05 to 2.0 MPaG. The top pressure is preferably from 0.1 to 1.0 MPaG, more preferably form 0.15 to 0.8 MPaG. If the top pressure is too low, a great cost is required for condensing the distillate conjugated diene at a low temperature, whereas if it is excessively high, the bottom temperature of the distillation column becomes high and the steam cost rises.

The bottom temperature is usually from 50 to 200° C., preferably from 80 to 180° C., more preferably from 100 to 160° C. If the bottom temperature is too low, distillation of the conjugated diene from the top becomes difficult, whereas if the temperature is excessively high, the solvent is also distilled from the top. The reflux ratio may be from 1 to 10 and is preferably from 2 to 4.

As the distillation column, either a packed column or a tray column may be used, and multistage distillation is preferred. For separating the conjugated diene and the solvent, the number of theoretical trays of the distillation column is preferably 5 or more, more preferably from 10 to 20. A distillation column exceeding 50 trays is not preferred in view of profitability of the distillation column construction, difficulty level of the operation, and safety control. Also, if the number of trays is too small, separation becomes difficult.

### (Purification Step)

A crude conjugated diene is obtained in the conjugated diene separation step, and a purification step of treating the crude conjugated diene by distillation/purification to make a further purified high-purity conjugated diene may be provided. The pressure at distillation of the distillation column used here may be arbitrarily set, but usually, the top pressure is preferably set to 0.05 to 0.4 MPaG. The top pressure is more preferably from 0.1 to 0.3 MPaG, still more preferably from 0.15 to 0.2 MPaG. If the top pressure is too low, a great cost is required for condensing the distillate conjugated diene at a low temperature, whereas if it is excessively high, the bottom temperature of the distillation column becomes high and the steam cost rises.

The bottom temperature is usually from 30 to 100° C., preferably from 40 to 80° C., more preferably from 50 to 60° C. If the bottom temperature is too low, distillation of the conjugated diene from the top becomes difficult, whereas if the temperature is excessively high, the amount of the conjugated diene condensed at the top is increased and the cost rises. The reflux ratio may be from 1 to 10 and is preferably from 2 to 4.

As the distillation column, either a packed column or a tray column may be used, and multistage distillation is preferred. For separating the conjugated diene and the impurity such as furan, the number of theoretical trays of the distillation column is preferably 5 or more, more preferably from 10 to 20. A distillation column exceeding 50 trays is not preferred in view of profitability of the distillation column construction, difficulty level of the operation, and safety control. Also, if the number of trays is too small, separation becomes difficult. The purified conjugated diene obtained in this way is a conjugated diene having a purity of 99.0 to 99.9%.

### [Mode for Carrying Out Process]

With respect to the mode for carrying out the process related to the production process of a conjugated diene of the present invention, a case of producing butadiene is described below by referring to the drawings.

FIG. 1 is one of the mode for carrying out the process of the present invention.

In FIG. 1, numeral 1 indicates a reactor (reaction column), 2 indicates a quench column, 3, 6 and 13 indicate a cooler (heat exchanger), 4, 7 and 14 indicate a drain pot, 8A and 8B indicate a dehydration column, 9 indicates a heater (heat exchanger), 10 indicates a solvent absorption column, 11 indicate a degassing column, 12 indicates a solvent separation column, and 100 to 126 indicate piping.

Incidentally, FIG. 1 shows a case where butene is used as BBSS and butadiene is used as the conjugated diene obtained.

n-Butene as a raw material or an n-butene-containing mixture such as BBSS is gasified in a vaporizer (not shown) and introduced via piping 101, and at the same time, a nitrogen gas, air (molecular oxygen-containing gas) and water (steam) are introduced via pipings 102, 103 and 104, respectively. The obtained mixed gas is heated to approximately from 150 to 400° C. in a preheater (not shown) and then supplied via piping 100 to a multitubular reactor 1 (oxidative dehydrogenation reactor) packed with a catalyst. The reaction product gas from the reactor 1 is fed to a quench column 2 via piping 105 and cooled to approximately from 20 to 99° C.

In the quench column 2, cooling water is introduced via piping 106 and counter-currently contacted with the product gas. The water after cooling the product gas by counter-current contact is discharged via piping 107. Incidentally, this cooling water effluent is cooled by a heat exchanger (not shown) and again circulated for utilization in the quench column 2.

The product gas cooled in the quench column 2 is distilled from the top and then cooled to room temperature through a cooler 3 via 108, and the condensed water generated by cooling is separated into a drain pot 4 via piping 109. The gas after separation of water further passes through piping 110 and is pressure-increased to approximately from 0.1 to 0.5 MPa by a compressor 5, and the pressure-increased gas passes through piping 111 and again cooled to approximately from 10 to 30° C. by a cooler 6. The condensed water generated by cooling is separated into a drain pot 7 via piping 112. The compressed gas after separation of water is introduced into dehydration columns 8A and 8B packed with a desiccant such as molecular sieve and dehydrated. In the dehydration columns 8A and 8B, dehydration of the compressed gas and regeneration of the desiccant by drying under heating are alternately performed. That is, the compressed gas is introduced into the dehydration column 8A via pipings 113 and 113a to be subjected to dehydration treatment and fed to a solvent absorption column 10 via pipings 114a and 114.

During this time, a nitrogen gas heated to approximately from 150 to 250° C. is introduced into a dehydration column 8B by passing through piping 122, a heater 9 and pipings 123, 123a and 123b, and desorption of water is effected by the heating of desiccant. The nitrogen gas containing the desorbed water passes through pipings 124a, 124b and 124 and is cooled to room temperature in a cooler 13 and after separating the condensed water into a drain pot 14 via piping 125, the gas is discharged via piping 126.

When the desiccant of the dehydration column 8A reaches saturation, the gas flow path is switched, and dehydration of the compressed gas is performed in the dehydration column 8B, and regeneration the desiccant in the dehydration column 8A is performed.

The desiccant regeneration time in the dehydration column in the dehydration step is not particularly limited but is usually from 6 to 48 hours, preferably from 12 o 36 hours, more preferably from 18 to 30 hours.

The dehydrated gas from the dehydration columns 8A and 8B is, if desired, cooled to approximately from 10 to 30° C. by a cooler (not shown), then fed to a solvent absorption column 10, and counter-currently contacted with a solvent (absorption solvent) introduced via piping 115. By this contact, the conjugated diene in the dehydrated gas and the unreacted raw material gas are absorbed by the absorption solvent. The component (off gas) unabsorbed by the absorption solvent is discharged via piping 117 from the top of the solvent absorption column 10 and discarded by burning. At this time, when a solvent having a relatively low boiling point such as toluene is used as the absorption solvent, the solvent is sometimes vaporized via piping 117 in an economically nonnegligible amount. In such a case, a step of recovering the low-boiling-point solvent by using a solvent having a higher boiling point may be provided ahead of piping 117. The solvent absorption liquid after letting butadiene or unreacted raw material gas be absorbed by the absorption solvent in the solvent absorption column 10 is withdrawn from the bottom of the solvent absorption column 10 and fed to an aeration column 11 via piping 116. In the solvent absorption liquid of butadiene obtained in the solvent absorption column 10, a slight amount of nitrogen or oxygen is also absorbed, and therefore, the solvent absorption liquid is supplied to the degassing column 11 and hated, whereby nitrogen or oxygen dissolved in the liquid is gasified and removed.

At this time, a part of the butadiene, the raw material gas and the solvent may be gasified, and therefore, the gas generated here is liquefied by a condenser (not shown) provided at the top of the degassing column 11 and recovered in the solvent absorption liquid. The raw material gas, butadiene and the like, which are not condensed, are withdrawn as a mixed gas of nitrogen and oxygen via piping 118 and for raising the recovery ratio of the conjugated diene, circulated to the inlet side of the compressor 5, and processing is again performed. On the other hand, the deaerated liquid resulting from degassing of the solvent absorption liquid is fed to a solvent separation column 12 via piping 119.

In the solvent separation column 12, distillation/separation of the conjugated diene is performed by a reboiler and a condenser, and a crude butadiene fraction is withdrawn via pining 120 from the top. The absorption solvent separated is withdrawn via piping 121 from the bottom and circulated for utilization as an absorption solvent in the solvent absorption column 10.

### EXAMPLES

### Production Example 1

### Preparation of Composite Oxide Catalyst

54 Gram of ammonium paramolybdate was dissolved in 250 ml of pure water under heating at 70° C. Separately, 7.18 g of ferric nitrate, 31.8 g of cobalt nitrate and 31.8 g of nickel nitrate were dissolved in 60 ml of pure water under heating at 70° C. These solutions were gradually mixed with thorough stirring.

Subsequently, 64 g of silica was added, and the mixture was thoroughly stirred. The resulting slurry was heated at 75° C. and ripened for 5 hours. Furthermore, the slurry was dried under heating and then heat-treated at 300° C. for 1 hour in an air atmosphere.

The obtained particulate solid (ignition loss: 1.4 wt %) of the catalyst precursor was ground, and 40.1 g of ammonium paramolybdate was dispersed in a solution obtained by adding and dissolving 10 ml of aqueous ammonia in 150 ml of pure water. Subsequently, 0.85 g of borax and 0.36 g of potassium nitrate were dissolved in 40 ml of pure water under heating at 25° C., and the resulting solution was added to the slurry above.

Furthermore, 58.1 g of bismuth subcarbonate containing 0.45% Na in the form of solid solution was added and mixed with stirring. The resulting slurry was dried by heating at 130° C. for 12 hours, and the obtained particulate solid was tablet-formed into a tablet of 5 mm in diameter and 4 mm in height by using a small molding machine and then calcined at 500° C. for 4 hours to obtain a catalyst. The catalyst was a composite oxide having the following atomic ratio as calculated from the charged raw materials.

Mo:Bi:Co:Ni:Fe:Na:B:K:Si=12:5:2.5:2.5:0.4:0.35:0.2:0.08:24

Also, the atomic proportions a₁ and a₂ of molybdenum at the preparation were 6.9 and 5.1, respectively.

### [Measurement of Explosion Range]

Mixed gases were prepared by variously changing the mixing ratio of nitrogen, air and combustible gas, and each mixed gas was introduced into a 1 L-volume pressure-resistant vessel equipped with a spark plug and a manometer, and whether the gas explodes or not was examined by striking sparks at the spark plug. The explosion was judged based on the following criteria, and the explosion range is determined using a combustible material concentration judged as no explosion or limit.

FIG. 2 shows the explosion range when the combustible gas is BBSS, and FIG. 4 shows the explosion range when the combustible gas is butadiene. Here, the rate of increase in explosion pressure was measured according to the formula: rate of increase in explosion pressure=(ΔP/P₀)×100 (ΔP=explosion pressure, P₀=pressure in the initial stage of measurement).

No explosion: The rate of increase in explosion pressure is less than 8%.

Limit: The rate of increase in explosion pressure is from more than 8% to less than 10%.

Explosion: The rate of increase in explosion pressure is more than 10%.

### Example 1

### Production of 1,3-Butadiene

Production of 1,3-butadiene was performed using the process shown in FIG. 1. Incidentally, for the analysis of gas in Examples, gas chromatography (GC-2014, manufactured by Shimadzu Corporation) was used.

In a reaction tube inside a reactor 1 equipped with 113 reaction tubes having an inner diameter of 27 mm and a length of 3,500 mm, 1,162 ml of the composite oxide catalyst produced in Production Example 1 and 407 ml of inert ball (produced by Tipton Corp.) were packed per one reaction tube. At this time, the catalytic layer was consisting of three layers, and the dilution ratios of the layers in the direction from the reactor inlet toward the product gas exit of the reactor were 60 vol %, 40 vol % and 0 vol %, respectively.

Also, out of the reaction tubes, a thermometer was disposed on three reaction tubes and measured the temperature in the reactor. Incidentally, the thermometer used was a multipoint thermocouple (manufactured by Okazaki Manufacturing Company) and measured the temperature distribution of the catalytic layer in the region from the inlet to the outlet of the reaction tube.

Also, air (molecular oxygen: 21%) and nitrogen (purity: 99.99% or more) were previously supplied to the reactor, and the temperature was raised by flowing a heating medium (dibenzyltoluene). After the temperature in the reactor reached 302° C., BBSS discharged in the process of extracting/separating butadiene from a C₄ fraction by-produced by naphtha cracking, air, nitrogen and steam were supplied at the following flow rates (per one reaction tube of the reactor) and mixed, and the mixture was heated to 217° C. by a preheater and then supplied to the reactor 1. FIG. 3 is a three-component diagram showing the state of combustible gas (BBSS) concentration in the mixed gas supplied to the reactor 1, where the explosion range of combustible gas (BBSS)-air-inert gas is indicated. An oxidative dehydrogenation reaction was performed in the reactor, and a butadiene-containing product gas exited from the reactor 1 outlet. In the periphery of the reaction tube in the reactor 1, a heating medium (dibenzyltoluene) at 319° C. was flowed to adjust the temperature inside the reaction tube to from 341 to 352° C.

BBSS: 13.2 parts by volume/hr

Air: 77.3 parts by volume/hr

Nitrogen: 28.5 parts by volume/hr

Steam: 22.4 parts by volume/hr

The composition of BBSS is as follows.

Propane: 0.035 mol %

Cyclopropane: 0.057 mol %

Propylene: 0.109 mol %

Isobutane: 4.784 mol %

n-Butane: 16.903 mol %

Trans-2-butene: 16.903 mol %

1-Butene: 43.487 mol %

Isobutene: 2.264 mol %

2,2-Dimethylpropane: 0.197 mol %

Cis-2-butene: 12.950 mol %

Isopentane: 0.044 mol %

n-Pentane: 0.002 mol %

1,2-Butadiene: 0.686 mol %

1,3-Butadiene: 1.075 mol %

Methyl acetylene: 0.017 mol %

3-Methyl-1-butene: 0.057 mol %

2-Pentene: 0.001 mol %

Vinyl acetylene: 0.006 mol %

Ethyl acetylene: 0.282 mol %

The product gas from the reactor 1 outlet was cooled to 86° C. by contacting it with water in a quench column 2 and further cooled to room temperature by a cooler 3. This gas was sampled and analyzed by gas chromatography, and the reaction results were a butene conversion of 95% and a butadiene selectivity of 86%.

The water condensed here was recovered in a drain pot 4. The gas was pressurized to 0.3 MPa by a compressor 5 and further cooled to about 17° C. by a cooler 6, and the water was thereby condensed and recovered in a drain pot 7.

The compressed gas was supplied to a dehydration column 8A or 8B packed with Molecular Sieve 3A (produced by Union Showa K.K.).

The dehydrated gas was supplied to a solvent absorption column 10 under a pressure of 0.2 MPaG at a temperature of 16° C., toluene as an absorption solvent was supplied at 600 kg/h to cause counter-current contact and absorb hydrocarbons such as butadiene, oxygen or nitrogen was then separated in a degassing column 11, and furthermore, 1,3-butadiene was separated from toluene in a solvent separation column 12 and recovered.

The gas supplied to the solvent absorption column 10 and the gas distilled from the top of the solvent absorption column 10 were sampled and analyzed, and the results were as follows.

Mixed Gas Supplied to Solvent Absorption Column 10:

Oxygen concentration: 6.1 vol % (29% in terms of air), and combustible gas concentration: 10.0 vol %.

### Product Gas Distilled From the Top of the Solvent Absorption Column 10:

Oxygen concentration: 6.8 vol % (32.4% in terms of air), and combustible gas concentration: 0.6 vol %.

These results are indicated in the three-component diagram showing the explosion range and, as shown in FIG. 5(a), it is revealed that even when the combustible gas is absorbed in the solvent absorption column, the composition does not traverse the explosion range. In FIG. 5(a), the oxygen concentration is shown in terms of air.

### Comparative Example 1

In one quartz-made reaction tube, 2 ml of the composite oxide catalyst produced in Production Example 1 and 2 ml of fused Al₂O₃ were packed. At this time, the catalytic layer was consisting of 2 layers, and the dilution ratios of layers in the direction from the inlet of the reactor to the product gas exit of the reactor were 66 vol % and 0 vol %, respectively.

Pure 1-butene, air and nitrogen were supplied at the following flow rates and mixed as a raw material gas, and the gas was supplied to the reaction tube. A thermocouple was inserted into the center of the reaction tube so that the reaction temperature can be measured, and the temperature was adjusted to 350° C. in an electric furnace.

1-Butene: 23.2 mmol/hr

Oxygen: 33.5 mmol/hr

Nitrogen: 126.0 mmol/hr

The product gas from the reaction tube was cooled to room temperature by a cooler and after separating the drain, analysis of the gas composition was performed by gas chromatography.

The reaction results were a butene conversion of 88%, a butadiene selectivity of 79%, an oxygen concentration of 11.1% (52.9% in terms of air), a combustible gas concentration of 14.6%, and nitrogen of 74.3%.

If this gas is contacted with toluene, the composition probably enters the explosion range and is dangerous and therefore, the solvent absorption test was abandoned.

Instead, the possibility of explosion was examined by comparison with the data of an explosion experiment performed in Reference Example. The data of Example 1 reveal that when the reaction gas is treated in a solvent absorption column 10, the combustible gas concentration becomes a substantially negligible concentration. Accordingly, the oxygen concentration is presumed to become:

11.1/(11.1+74.3)×100=13.0% (61.9% in terms of air)

These results are indicated in the three-component diagram showing the explosion range of combustible gas (butadiene)-air-inert gas and, as shown in FIG. 5(b), it is revealed that as a result of the combustible gas (butadiene) in the product gas being absorbed in the absorption column, the composition traverses the explosion range.

In FIG. 5(b), the oxygen concentration is shown in terms of air.

### Example 2

### Adjustment of Oxygen Concentration

The process was performed in the same manner as in Example 1 except for changing the supply amounts of raw materials and the temperatures of preheater and heating medium as follows. FIG. 3 is a three-component diagram showing the state of combustible gas (BBSS) concentration in the mixed gas supplied to the reactor 1, where the explosion range of combustible gas (BBSS)-air-inert gas is indicated.

BBSS: 12.7 parts by volume/hr

Air: 69.6 parts by volume/hr

Nitrogen: 36.1 parts by volume/hr

Steam: 22.6 parts by volume/hr

Temperature of preheater for raw materials 219° C.

Temperature of heating medium 321.3° C.

The catalytic layer reached a temperature of 335 to 352° C.

The oxygen concentration of the reaction gas was measured by an oximeter in a magnetic dumbbell system provided behind the cooler 3 and found to be 5.0%. The operation was continued by setting the target oxygen concentration to 5.0%, but after 18 hours, the oxygen concentration was raised to 5.2%. The operation conditions were not changed, but it is considered that the composition of BBSS or the activity of catalyst was fluctuated.

Therefore, the present temperature of the apparatus for heating a heating medium was raised by 1° C., as a result, the temperature of the heating medium became 322.2° C. and the oxygen concentration was returned to 5.0%. FIG. 6(a) shows details of the change here in the oxygen concentration and heating medium temperature.

It is revealed from the results that the oxygen concentration of the product gas can be controlled by changing the heating medium temperature.

### Example 3

### Adjustment of Oxygen Concentration

The process was performed in the same manner as in Example 1 except for changing the supply amounts of raw materials and the temperatures of preheater and heating medium as follows. FIG. 3 is a three-component diagram showing the state of combustible gas (BBSS) concentration in the mixed gas supplied to the reactor 1, where the explosion range of combustible gas (BBSS)-air-inert gas is indicated.

BBSS: 12.7 parts by volume/hr

Air: 69.8 parts by volume/hr

Nitrogen: 36.1 parts by volume/hr

Steam: 22.4 parts by volume/hr

Temperature of preheater for raw materials 219° C.

Temperature of heating medium 319.7° C.

The catalytic layer reached a temperature of 332 to 350° C.

The oxygen concentration of the reaction gas was measured by an oximeter in a magnetic dumbbell system provided behind the cooler 3 and found to be 5.4%. The operation was continued by setting the target oxygen concentration to 5.4%, but after 26 hours, the oxygen concentration was reduced to 5.2%. The operation conditions were not changed, but it is considered that the composition of BBSS or the activity of catalyst was fluctuated.

Therefore, the present temperature of the apparatus for heating a heating medium was lowered by 1° C., as a result, the temperature of the heating medium became 318.3° C. and the oxygen concentration was returned to 5.4%. FIG. 6(b) shows details of the change here in the oxygen concentration and heating medium temperature.

### Example 4

### Adjustment of Oxygen Concentration

The process was performed in the same manner as in Example 1 except for changing the supply amounts of raw materials and the temperatures of preheater and heating medium as follows. FIG. 3 is a three-component diagram showing the state of combustible gas (BBSS) concentration in the mixed gas supplied to the reactor 1, where the explosion range of combustible gas (BBSS)-air-inert gas is indicated.

BBSS: 13.2 parts by volume/hr

Air: 70.1 parts by volume/hr

Nitrogen: 36.0 parts by volume/hr

Steam: 22.5 parts by volume/hr

Temperature of preheater for raw materials 217.8° C.

Temperature of heating medium 322.5° C.

The catalytic layer temperature was from 339 to 354° C., and the oximeter provided behind the cooler 3 indicated 4.7%. In the following, the target oxygen concentration was set to 4.7%. The reaction results were a butene conversion of 93% and a butadiene selectivity of 89%.

The heating medium temperature was changed to 329° C. for raising the butene conversion, as a result, the reaction results were a butene conversion of 96% and a butadiene selectivity of 84%. However, the oximeter indicated 3.6% which was lower than the target oxygen concentration. Therefore, the flow rate of air supplied to the reactor was increased to 80 parts by volume/hr and for keeping the total flow rate of raw materials from changing, the flow rate of nitrogen was decreased to 26 parts by volume/hr, as a result, the oximeter indicated 4.6% which is almost the target.

It is revealed from the results that the oxygen concentration of the product gas can be controlled also by changing the supply amount of air.

### Example 5

In a stainless steel-made reaction tube having an inner diameter of 23.0 mm and a length of 500 mm, 20.0 ml of the composite oxide catalyst produced in Production Example 1 and 20.0 ml of inert ball (produced by Tipton Corp.) were packed after mixing them, whereby the dilution ratio of the catalytic layer was set to 50 vol %.

An insertion tube having an outer diameter of 2.0 mm was disposed in the reaction tube, and by disposing a thermocouple in the insertion tube, the temperature in the reactor was measured. As the heating medium, an electric furnace was used.

Nitrogen at 12.9 L/hr, air at 16.2 L/hr, and steam at 14.3 L/hr were previously supplied to a preheater, and thereafter, BBSS at 3.6% which is the raw material gas having a composition shown in Table 1, was supplied. These were mixed in the preheater, and the resulting mixed gas was heated to 335° C. (composition of the mixed gas introduced into the reactor=nitrogen: 27.4 vol %, air: 34.5 vol %, steam: 30.5 vol %, raw material gas: 7.6 vol %). A representative composition (mol %) of components contained in BBSS as the raw material gas is shown in Table 1. At this time, the flow rate of the mixed gas was 47.0 L/h, and the ratio of the amount of catalyst and the flow rate of mixed gas in the reactor was 2,350 h⁻¹. FIG. 3 is a three-component diagram showing the state of combustible gas (BBSS) concentration in the mixed gas supplied to the reaction tube, where the explosion range of combustible gas (BBSS)-air-inert gas is indicated.

An oxidative dehydrogenation reaction was performed by supplying the mixed gas to the reactor. The temperature of the catalytic layer in the reactor was 354° C. on average, and the pressure was 2 kPa as the gauge pressure. The product gas from the reactor outlet was cooled by a cooling tube having disposed therein a filter, further cooled by contacting the gas with water, and analyzed by gas chromatography (Model No. GC-8A, GC-9A, manufactured by Shimadzu Corporation). The oxygen concentration in the product gas was 7.2 vol %.

The n-butene conversion (conversion in total of 1-butene, cis-2-butene and trans-2-butene) was 79.6 mol %, and the butadiene selectivity was 92.6 mol %. After 8 hours, the reaction was stopped. The amount of solid byproducts caught in the filter inside the cooling tube was 38.9 mg, and the production amount of solid byproducts per 1 hour was 4.6 mg/h. The production amount of butadiene was 4,529 mg/h, and the production amount of solid matters was 0.10 wt % based on the production amount of butadiene. The results are shown in Table 1.

### Example 6

The process was performed under the same conditions as in [Example 5] except for performing the oxidative dehydrogenation reaction by setting the temperature of the catalytic layer in the reactor to 357° C. on average. FIG. 3 is a three-component diagram showing the state of combustible gas (BBSS) concentration in the mixed gas supplied to the reaction tube, where the explosion range of combustible gas (BBSS)-air-inert gas is indicated. The oxygen concentration in the product gas was 6.6 vol %. The results are shown in Table 1.

### Example 7

The process was performed under the same conditions as in [Example 5] except for supplying nitrogen at 18.9 L/hr, air at 13.1 L/hr, steam at 11.2 L/hr and BBSS as the raw material gas at 3.6 L/hr. FIG. 3 is a three-component diagram showing the state of combustible gas (BBSS) concentration in the mixed gas supplied to the reaction tube, where the explosion range of combustible gas (BBSS)-air-inert gas is indicated. The oxygen concentration in the product gas was 4.5 vol %. The results are shown in Table 1.

### Example 8

In a stainless steel-made reaction tube having an inner diameter of 23.0 mm and a length of 500 mm, 24 ml of inert ball (size per particle: about 0.065 mm³) was previously packed (packed layer length: 210 mm), and only 20.0 ml of the composite oxide catalyst produced in Production Example 1 was packed on the inert ball packed layer, whereby the dilution ratio of the catalytic layer was set to 0 vol %.

An insertion tube having an outer diameter of 2.0 mm was disposed in the reaction tube, and by putting a sheath type thermocouple (manufactured by Takahashi Thermosensor) in the insertion tube, the temperatures in the reactor (temperature at the outlet of the catalytic layer, highest temperature of catalytic layer) were measured. As the heating medium, an electric furnace was used.

Nitrogen at 7.8 L/hr, air at 16.0 L/hr, and steam at 5.5 L/hr were previously supplied to a preheater, and thereafter, BBSS as the raw material gas at 2.8 L/hr was supplied. These were mixed in the preheater, and the resulting mixed gas was heated to 345° C. A representative composition (mol %) contained in the raw material gas is shown in Table 1.

Subsequently, an oxidative dehydrogenation reaction was performed by continuously supplying the mixed gas at 32.1 L/hr from the top of the reaction tube, and the product gas was withdrawn from the bottom of the reaction tube. The ratio of the amount of catalyst and the flow rate of mixed gas in the reactor was 1,400 h⁻¹. FIG. 3 is a three-component diagram showing the state of combustible gas (BBSS) concentration in the mixed gas supplied to the reaction tube, where the explosion range of combustible gas (BBSS)-air-inert gas is indicated.

The temperature of the catalytic layer in the reaction tube was 374° C. on average, and the pressure was 2 kPa as the gauge pressure. Also, the highest temperature in the reaction tube was 387° C. The product gas from the reactor outlet was cooled by a cooling tube having disposed therein a filter, further cooled by contacting the gas with water, and analyzed by gas chromatography (Model No. GC4000, manufactured by GL Sciences). The oxygen concentration in the product gas was 4.8 vol %.

The n-butene conversion (conversion in total of 1-butene, cis-2-butene and trans-2-butene) was 91.4 mol %, and the butadiene selectivity was 89.0 mol %. The reaction was stopped 200 hours after BBSS as the raw material gas was supplied. All catalysts were withdrawn from the reaction tube, and the amount of carbon attached to the withdrawn catalysts was measured (measurement apparatus: carbon-sulfur analyzer, Model No. CS600, manufactured by LECO), as a result, the carbon concentration was 2.1 wt % (increase in the concentration of carbon attached to catalyst particle between before and after reaction: 0.6 wt %). The results are shown in Table 1.

### Example 9

In [Example 8], 23.0 ml of the composite oxide catalyst produced in Production Example 1 and 23.0 ml of inert ball (size per particle: about 0.065 mm³) were mixed and packed, whereby the dilution ratio of the catalytic layer was set to 50 vol %.

The process was performed under the same conditions except for supplying nitrogen at 10.9 L/hr, air at 12.9 L/hr, steam at 5.5 L/hr, and BBSS as the raw material gas at 2.8 L/hr. FIG. 3 is a three-component diagram showing the state of combustible gas (BBSS) concentration in the mixed gas supplied to the reaction tube, where the explosion range of combustible gas (BBSS)-air-inert gas is indicated. The oxygen concentration in the product gas was 3.5 vol %. The results are shown in Table 1.

### Comparative Example 2

The process was performed under the same conditions as in [Example 5] except for mixing 10.0 ml of the composite oxide catalyst produced in Production Example 1 and 10.0 ml of inert ball (produced by Tipton Corp.), packing the mixture to provide a catalytic layer, and supplying nitrogen at 3.6 L/hr, air at 10.9 L/hr, steam at 7.2 L/hr, and BBSS as the raw material gas at 1.8 L/hr. FIG. 3 is a three-component diagram showing the state of combustible gas (BBSS) concentration in the mixed gas supplied to the reaction tube, where the explosion range of combustible gas (BBSS)-air-inert gas is indicated. The oxygen concentration in the product gas was 8.1 vol %. The results are shown in Table 1.

### Comparative Example 3

In [Example 8], 20.0 ml of the composite oxide catalyst produced in Production Example 1 and 20.0 ml of inert ball (size per particle: about 0.065 mm³) were mixed and packed, whereby the dilution ratio of the catalytic layer was set to 50 vol %.

The process was performed under the same conditions except for supplying nitrogen at 11.1 L/hr, air at 9.6 L/hr, steam at 4.8 L/hr, and BBSS as the raw material gas at 2.5 L/hr. FIG. 3 is a three-component diagram showing the state of combustible gas (BBSS) concentration in the mixed gas supplied to the reaction tube, where the explosion range of combustible gas (BBSS)-air-inert gas is indicated. The oxygen concentration in the product gas was 2.0 vol %. The results are shown in Table 1.

### [Results]

Comparison of Examples 5 to 7 with Comparative Example 2 reveals that when the oxygen concentration in the product gas is controlled to 8.0 vol % or less, the production amount of byproduct solid matters based on the production amount of butadiene is reduced.

Also, comparison of Examples 8 and 9 with Comparative Example 3 reveals that when the oxygen concentration in the product gas is controlled to 2.5 vol % or more, for example, attachment of carbon portion on catalyst (coking) is suppressed.

That is, when the oxygen concentration in the product gas is from 2.5 to 8.0 vol %, the production amount of high-boiling-point byproducts precipitated in the cooling step after the reaction step can be reduced and at the same time, coking of a carbon content or the like on the catalyst can be prevented from proceeding.

It is understood from these results that in the industrial process, the differential pressure of the reactor can be kept from rising in course of long-term operation, generation of a trouble due to clogging or the like can be also suppressed, and butadiene can be stably produced.

TABLE 1

| | | | | | | Comparative | | | Comparative |
|---|---|---|---|---|---|---|---|---|---|
| | | | Example 5 | Example 6 | Example 7 | Example 2 | Example 8 | Example 9 | Example 3 |
| Catalyst dilution ratio of catalytic | | (vol %) | 50 | 50 | 50 | 50 | 0 | 50 | 50 |
| layer | | | | | | | (no dilution) | | |
| Supply amount of nitrogen | | (L/hr) | 12.9 | 12.9 | 18.9 | 3.6 | 7.8 | 10.9 | 11.1 |
| Supply amount of air | | (L/hr) | 16.2 | 16.2 | 13.1 | 10.9 | 16.0 | 12.9 | 9.6 |
| Supply amount of steam | | (L/hr) | 14.3 | 14.3 | 11.2 | 7.2 | 5.5 | 5.5 | 4.8 |
| Supply amount of BBSS | | (L/hr) | 3.6 | 3.6 | 3.6 | 1.8 | 2.8 | 2.8 | 2.5 |
| n-Butene in BBSS | | (L/hr) | 2.6 | 2.6 | 2.6 | 1.3 | 2.0 | 2.0 | 2.0 |
| Composition of | 1-butene | (mol %) | 42.0 | 42.0 | 42.0 | 42.0 | 42.0 | 42.0 | 42.0 |
| BBSS | cis-2-butene | (mol %) | 12.7 | 12.7 | 12.7 | 12.7 | 12.7 | 12.7 | 12.7 |
| | trans-2-butene | (mol %) | 16.1 | 16.1 | 16.1 | 16.1 | 16.1 | 16.1 | 16.1 |
| | others | (mol %) | 29.2 | 29.2 | 29.2 | 29.2 | 29.2 | 29.2 | 29.2 |

| | | | | | | | | |
|---|---|---|---|---|---|---|---|---|
| Flow rate of mixed gas | (L/hr) | 47 | 47 | 47 | 23.5 | 32.1 | 32.1 | 28 |
| BBSS Concentration in mixed gas | (vol %) | 7.6 | 7.6 | 7.6 | 7.6 | 8.8 | 8.8 | 8.8 |
| Ratio of flow rate of mixed gas to | (h⁻¹) | 2350 | 2350 | 2350 | 2350 | 1400 | 1400 | 1400 |
| catalyst amount | | | | | | | | |
| n-Butene conversion | (%) | 79.6 | 81.9 | 82.1 | 79.8 | 91.4 | 90.4 | 88.8 |
| Butadiene selectivity | (%) | 92.6 | 93.6 | 93.6 | 92.0 | 89.0 | 87.9 | 88.2 |
| Oxygen concentration in product gas | (vol %) | 7.2 | 6.6 | 4.5 | 8.1 | 4.8 | 3.5 | 2.0 |
| Carbon composition of catalyst | (wt %) | | | | | 0.6 | 2.3 | 3.1 |
| withdrawn | | | | | | | | |
| Amount of byproduct solid matter | (mg/hr) | 4.6 | 3.0 | 3.5 | 7.7 | | | |
| Production amount of butadiene | (mg/hr) | 4529 | 4533 | 4465 | 2023 | | | |
| Production amount of byproduct solid | (wt %) | 0.10 | 0.07 | 0.08 | 0.38 | | | |
| matter based on production amount of | | | | | | | | |
| butadiene | | | | | | | | |

While the invention has been described in detail and with reference to specific embodiments thereof, it will be apparent to one skilled in the art that various changes and modifications can be made therein without departing from the spirit and scope of the invention. This application is based on Japanese Patent Application (Patent Application No. 2009-131147) filed on May 29, 2009, the contents of which are incorporated herein by way of reference.

### INDUSTRIAL APPLICABILITY

According to the present invention, in producing a conjugated diene by an oxidative dehydrogenation reaction of a monoolefin having a carbon atom number of 4 or more, accumulation of a carbon portion such as coke on the catalyst in the reactor can be suppressed, the production amount of high-boiling-point byproducts which precipitate in the cooling step after the reaction step can be reduced, and stable operation of the plant can be more safely and continuously performed.

### DESCRIPTION OF REFERENCE NUMERALS AND SIGNS

1 Reactor (reaction column)
2 Quench column
3, 6, 13 Cooler
4, 7, 14 Drain pot
5 Compressor
8A, 8B Dehydration column
9 Heater (heat exchanger)
10 Solvent absorption column
11 Degassing column
12 Solvent separation column
31 Evaporation column
32 First extractive distillation column
33 i-Butene separation column
34 Preliminary stripping column
35 First stripping column
36 Compressor
37 Second extractive distillation column
38 Butadiene recovery column
39 Second stripping column
40 First distillation column
41 Second distillation column
100 to 126 Piping
200 to 219 Piping

1. A production process of a conjugated diene, comprising a step of mixing a raw material gas containing a monoolefin having a carbon atom number of 4 or more and a molecular oxygen-containing gas and supplying the mixture to a reactor, and a step of obtaining a corresponding conjugated diene-containing product gas produced by an oxidative dehydrogenation reaction of the monoolefin having a carbon atom number of 4 or more in the presence of a catalyst, wherein the concentration of a combustible gas in the gas supplied to the reactor is not less than the upper explosion limit and the oxygen concentration in the product gas is from 2.5 to 8.0 vol %.

2. The production process of a conjugated diene as claimed in claim 1, which further comprises a step of bringing the conjugated diene-containing product gas into contact with an absorption solvent to obtain a conjugated diene-containing solvent.

3. The production process of a conjugated diene as claimed in claim 1 or 2, wherein the catalyst is a composite oxide catalyst containing at least molybdenum, bismuth and cobalt.

4. The production process of a conjugated diene as claimed in claim 3, wherein the catalyst is a composite oxide catalyst represented by the following formula (1):
MoₐBi_{b}Co_{c}Ni_{d}FeₑX_{f}Y_{g}ZₕSiᵢOⱼ (1)
(wherein X is at least one element selected from the group consisting of magnesium (Mg), calcium (Ca), zinc (Zn), cerium (Ce) and samarium (Sm), Y is at least one element selected from the group consisting of sodium (Na), potassium (K), rubidium (Rb), cesium (Cs) and thallium (Tl), Z is at least one element selected from the group consisting of boron (B), phosphorus (P), arsenic (As) and tungsten (W), a to j represent an atomic ratio of respective elements and when a=12, are in ranges of b=0.5 to 7, c=0 to 10, d=0 to 10 (provided that c+d=1 to 10), e=0.05 to 3, f=0 to 2, g=0.04 to 2, h=0 to 3 and i=5 to 48, and j is a numerical value satisfying the oxidation state of other elements).

5. The production process of a conjugated diene as claimed in claim 4, wherein the composite oxide catalyst is a catalyst produced through a step including integration in an aqueous system and heating of supply source compounds of respective component elements constituting the composite oxide catalyst and is produced by a method comprising a pre-step of producing a catalyst precursor by heat-treating an aqueous solution or aqueous water dispersion of the raw material compound containing silica and at least one member selected from the group consisting of a molybdenum compound, an iron compound, a nickel compound and a cobalt compound, or a dry matter resulting from drying of the aqueous solution or aqueous water dispersion, and a post-step of integrating the catalyst precursor, a molybdenum compound and a bismuth compound together with an aqueous solvent, and drying and firing the mixture.

6. The production process of a conjugated diene as claimed in claim 1, wherein the oxygen concentration of the product gas is measured at the outlet of the reactor and at least either one of the amount of the molecular oxygen-containing gas supplied to the reactor or the reactor temperature is controlled according to the oxygen concentration, thereby keeping the oxygen concentration in the product gas to a range of 2.5 to 8 vol %.

7. The production process of a conjugated diene as claimed in claim 1, wherein the raw material gas is a gas containing 1-butene, cis-2-butene, trans-2-butene or a mixture thereof obtained by dimerization of ethylene, or a gas containing hydrocarbons having a carbon atom number of 4 obtained when fluid catalytically cracking a heavy oil fraction or a butene fraction produced by dehydrogenation or oxidative dehydrogenation reaction of n-butane.
